(19) United States
(12) Patent Application Publication
(10) Pub. No.: US 2024/0190802 A1
(43) Pub. Date: Jun. 13, 2024

(54) DIRECT AND SCALABLE SYNTHESIS OF SOLUTION PROCESSABLE METAL-ORGANIC FRAMEWORK NANOSHEETS WITH VARIABLE FUNCTIONALITIES

(71) Applicant: National University of Singapore, Singapore (SG)

(72) Inventors: Dan Zhao, Singapore (SG); Hongye Yuan, Singapore (SG)

(73) Assignee: National University of Singapore, Singapore (SG)

(21) Appl. No.: 18/286,717

(22) PCT Filed: Apr. 7, 2022
(86) PCT No.: PCT/SG2022/050201

§ 371 Date: Oct. 12, 2023

(30) Foreign Application Priority Data
Apr. 12, 2021 (SG) 10202103693W

(51) Int. Cl. C07C 51/41; B01J 13/00

(52) U.S. Cl. CPC C07C 51/418; B01J 13/0021

(57) ABSTRACT

Disclosed herein are metal organic framework (MOF) suspensions, and sols and compositions comprising MOF nanosheets. Also disclosed herein are their methods of preparation.

### FIELD OF INVENTION

The current invention relates to the synthesis of metal-organic framework (MOF) suspension, and solution processable MOF nanosheets with variable functionalities in a direct and scalable manner. The current invention also relates to suspensions, sols and compositions of MOF nanosheets and their preparation methods.

### BACKGROUND

The listing or discussion of a prior-published document in this specification should not necessarily be taken as an acknowledgement that the document is part of the state of the art or is common general knowledge.

The synthesis of porous materials that combine the solution processability of polymers with permanent porosity remains challenging but highly attractive for many applications, including molecular sieving technologies, drug delivery, and electronics. Recently, noteworthy successes have been reported in the design and synthesis of porous organic cages and metal-organic polyhedrons (MOPs) with well-defined pore space and high solubility/dispersity in bulky solvents. Metal-organic frameworks (MOFs), constructed by discrete inorganic secondary building units (SBUs) and organic linkers, have emerged as a novel class of crystalline porous materials featuring high degrees of designability in porosity, structural topology, and host-guest interactions (S. Kitagawa, R. Kitaura & S.-i. Noro, Angew. Chem. Int. Ed. 2004, 43, 2334; H.-C. Zhou, J. R. Long & O. M. Yaghi, Chem. Rev. 2012, 112, 673; and G. Maurin et al., Chem. Soc. Rev. 2017, 46, 3104). Typically, their pore size and shape, dimensionality, and active sites (e.g. appended functional groups, coordinatively unsaturated open metal sites) can be modulated judiciously via isoreticular chemistry. The past decade has witnessed significant advances in the synthesis of novel MOFs with adjustable physicochemical properties and their applications in molecule storage/separation (J.-R. U, R. J. Kuppler & H.-C. Zhou, Chem. Soc. Rev. 2009, 38, 1477), catalysis (J. Liu et al., Chem. Soc. Rev. 2014, 43, 6011), drug delivery (W. Cai et al., Adv. Sci. 2019, 6, 1801526), and chemical sensing (W. P. Lustig et al., Chem. Soc. Rev. 2017, 46, 3242). However, MOFs intrinsically lack fluidity and thus processability. This issue severely impedes the scale-up production of MOF-based devices such as membranes and electronic devices, thus restricting their industrial applications.

The challenge associated with the poor solution processability of MOFs arises from their insoluble nature due to the extended networks with crystallinity and somehow structural rigidity (A. Bavykina, A. Cadiau & J. Gascon, Coord. Chem. Rev. 2019, 386, 85). Precipitation and agglomeration of MOFs ubiquitously happen once formed. Various strategies have been reported so far to alleviate the issue above. One straightforward method concerns the direct dispersion of limited choices of MOFs within hindered ionic liquids that are too bulky to enter the pores of MOFs (M. Costa Gomes et al., Angew. Chem. Int. Ed. 2018, 57, 11909). Stable suspensions exhibiting high gas adsorption capabilities can thus be afforded. Alternatively, MOF particles decorated by alkyl or carbene chains as stabilizing groups can also be well dispersed and stabilized in bulky solvents (S. He et al., J. Am. Chem. Soc. 2019, 141, 19708; and A. Knebel et al., Nat. Mater. 2020, 19, 1346). Therefore, the resultant so-called “porous liquids” combining characteristics of permanent porosity and fluidity could be utilized as liquid adsorbents or facilely processed as membranes with salient separation performance. Nevertheless, this strategy involves delicate functionalization toward MOF particles, and the installation of organic molecules undoubtedly introduces nonactive species, which may block the pores of MOFs and are detrimental to the physicochemical performance of relevant MOFs.

Dissimilarly, few MOFs with melting behavior can exceptionally form a glassy phase with certain fluidity under heating in an inert atmosphere (D. Umeyama et al., J. Am. Chem. Soc. 2015, 137, 864; T. D. Bennett et al., Nat. Commun. 2015, 6, 8079; and R. Gaillac et al., Nat. Mater. 2017, 16, 1149). Under this top-down event, the molten MOFs inherit the primary coordinative network and porosity of the original MOFs while forfeiting their long-range ordering. Obviously, one-pot and bottom-up synthesis of MOFs with fluidic features resembling liquid-crystalline (LC) materials is highly sought-after but remains rudimentary. Despite a handful of studies reporting the sporadic synthesis of colloidal and metastable solutions containing ultra-small three-dimensional (3D) MOF nanoparticles (typically below 50 nm) under modulated conditions (A. K. Chaudhari, I. Han & J.-C. Tan, Adv. Mater. 2015, 27, 4438; B. Bueken et al., Chem. Sci. 2017, 8, 3939; and J. Hou, A. F. Sapnik & T. D. Bennett, Chem. Sci. 2020, 11, 310), the solution processability of those MOFs into large-area films with a regular arrangement of nanochannels/cavities has not been demonstrated yet. In addition, two-dimensional (2D) MOF nanosheets with a high aspect ratio, precisely tunable thickness from single layer to multiple layers, and more readily accessible active sites frequently exhibit exotic physicochemical properties conspicuously distinct from those of their bulk counterparts (R. Makiura et al., Nat. Mater. 2010, 9, 565; S. Motoyama et al., J. Am. Chem. Soc. 2011, 133, 5640; S. Sakaida et al., Nat. Chem. 2016, 8, 377; and X. Wang et al., Nat. Commun. 2017, 8, 14460). Methodologies in producing free-standing MOF nanosheets include top-down physical or chemical exfoliation from their layered 3D counterparts and direct bottom-up synthesis (T. Rodenas et al., Nat. Mater. 2015, 14, 48; M. Zhao et al., Chem. Soc. Rev. 2018, 47, 6267; D. J. Ashworth & J. A. Foster, J. Mater. Chem. A 2018, 6, 16292; Y.-Z. Li, Z.-H. Fu & G. Xu, Coord. Chem. Rev. 2019, 388, 79; S. Zhao et al., Nat. Energy 2016, 1, 16184; and L. Cao et al., Angew. Chem. Int. Ed. 2016, 55, 4962). However, there are only a few reports in the direct and scalable synthesis of large-area MOF nanosheets with uniform thickness (M. Zhao et al., Chem. Soc. Rev. 2018, 47, 6267; and Y.-Z. Li, Z.-H. Fu & G. Xu, Coord. Chem. Rev. 2019, 388, 79). Although the interfacial-mediated and Langmuir-Blodgett methods allow the formation of large-area nanosheets, their scaling-up is problematic. This certainly limits the feasibility of MOF nanosheets in applications requiring large-area fabrication, such as electronics and membrane-based separation.

Therefore, there is a need to discover new methods for direct and scalable synthesis of large-area and solution processable MOF nanosheets.

### SUMMARY OF INVENTION

1. A method of forming a metal-organic framework (MOF) suspension, the process comprising:
(a) providing a metal precursor, an organic ligand, a capping molecule and a solvent comprising a polar aprotic solvent and water;
(b) adding the metal precursor, organic ligand and capping molecule to the solvent to provide a reaction mixture and heating it for a period of time to form the MOF suspension, wherein:
   the metal precursor has a concentration in the solvent of from 1.0 to 2 mg/mL; the organic ligand has a concentration in the solvent of from 1.0 to 2 mg/mL;
   the capping molecule is provided in an amount of from 5 to 20 vol % relative to the total volume of the solvent, and/or it is provided in an amount of from 5 to 20 wt % relative to the total weight of the solvent; and
   water represents from 5 to 20 vol % of the solvent, with the balance being the polar aprotic organic solvent.
2. The method according to Clause 1 further comprising the purification steps of:
(a) removing the MOF from the reaction mixture by centrifugation;
(b) adding a polar aprotic solvent to the removed MOF to form a suspension and then removing the MOF by centrifugation;
(c) repeating step (b) from one to ten times; and
(d) adding water to the removed MOF to form a suspension and then removing the MOF by centrifugation and repeating from two to ten times (e.g. from 4 to 6 times, such as five times) over a 24-hour period;
(e) adding water and/or tetrahydrofuran to the removed MOF to provide a final suspension of the MOF.
3. The method according to Clause 1 or Clause 2, wherein the polar aprotic solvent is selected from one or more of N,N-dimethylformamide (DMF), acetone, acetonitrile, dimethyl sulfoxide, dimethylacetamide, N,N-diethylformamide, optionally wherein the polar aprotic solvent is selected from one or more of N,N-dimethylformamide, dimethylacetamide, and N,N-diethylformamide (e.g. the polar aprotic solvent is N,N-dimethylformamide).
4. The method according to any one of the preceding clauses, wherein the capping molecule is provided in an amount of from 10 to 15 vol % relative to the total volume of the solvent, and/or it is provided in an amount of from 10 to 15 wt % relative to the total weight of the solvent, optionally wherein the capping molecule is provided in an amount of about 11.8 vol % relative to the total volume of the solvent, and/or it is provided in an amount of about 15 wt % relative to the total weight of the solvent.
5. The method according to any one of the preceding clauses, wherein the metal precursor is selected from one or more of ZrOX₂, HfOX₂, Zr₄, HfX₄, Alk, Crk, Feh, and Ti₄, where X is halo selected from Cl, Br, I, or F, optionally wherein the metal precursor is selected from one or more of ZrX₄ and HfX₄ (e.g. the metal precursor is ZrCl₄ or HfCl₄).
6. The method according to any one of the preceding clauses, wherein the organic ligand is a tridentate carboxylic acid that does not incorporate further functional groups or is a tridentate carboxylic acid that incorporates further functional groups (e.g. from 1 to 10, such as from 1 to 5, such as from 1 to 3 further functional groups), optionally wherein the organic ligand is selected from one or more of benzene-1,3,5-tribenzoate, 5′-(4-carboxyphenyl)-2′-methyl[1,1′:3′,1″-terphenyl]-4,4″-dicarboxylic acid, 2′-amino-5′-(4-carboxyphenyl)-[1,1′:3′,1″-terphenyl]-4,4″-dicarboxylic acid, 4,4′,4″-(1,3,5-triazine-2,4,6-triyl)tribenzoic acid, and 4,4′,4″-(pyridine-2,4,6-triyl)tribenzoic acid.
7. The method according to any one of the preceding clauses, wherein the capping molecule is a monodentate carboxylic acid, optionally wherein the capping ligand is selected from one or more of acetic acid, formic acid, and other monodentate acids, for example, the capping molecule is selected from acetic acid and/or formic acid (e.g. the capping molecule is formic acid).
8. The method according to any one of the preceding clauses, wherein one or both of the following apply:
(a) the metal precursor has a concentration in the solvent of from 1.5 to 1.8 mg/mL, such as about 1.76 mg/mL;
(b) the organic ligand has a concentration in the solvent of from 1.5 to 1.8 mg/mL, such as about 1.76 mg/mL;
(c) the period of time in step (b) of Clause 1 is from 1 hour to 72 hours, such as about 24 hours;
(d) the heating applied to the reaction mixture in step (b) of Clause 1 is from 100 to 150° C., such as about 120° C.
9. An aqueous and/or tetrahydrofuran suspension (e.g. a colloidal suspension) of a metal organic framework comprising:
   water and/or tetrahydrofuran (THF); and
   MOF nanosheets having an area of greater than or equal to 10,000 μm².
10. A sol comprising:
   a solvent (e.g. selected from one or more of water, THF and DMF); and
   MOF nanosheets having an area of greater than or equal to 10,000 μm².
11. A composition comprising MOF nanosheets having an area of greater than or equal to 10,000 μm².
12. The suspension according to Clause 9, the sol according to Clause 10 or the composition according to Clause 11, wherein the MOF nanosheets have an area of from 10,000 to 30,000 μm², such as from 15,000 to 29,000 μm², such as from 18,000 to 25,000 μm².
13. The suspension according to Clause 9 or Clause 12, the sol according to Clause 10 or Clause 12, or the composition according to Clause 11 or Clause 12, wherein the MOF in the MOF nanosheets is selected from one or more of NUS-8 and/or an analogue thereof, where each MOF is formed from:
(a) a metal ion and an organic ligand; or
(b) a metal ion and an organic ligand and a capping molecule.
14. The suspension according to any one of Clause 9, or Clauses 12 to 13, the sol according to any one of Clause 10 or Clauses 12 to 13, or the composition according to any one of Clauses 11 to 13, wherein the metal ion is selected from Zr⁴⁺, Hf⁴⁺, Al³⁺, Cr³⁺, Fe³⁺, and Ti⁴⁺, optionally wherein the metal ion is selected from one or more of Zr⁴⁺ and Hf⁴⁺.
15. The suspension according to any one of Clause 9, or Clauses 12 to 14, the sol according to any one of Clause 10 or Clauses 12 to 14, or the composition according to any one of Clauses 11 to 14, wherein the organic ligand is a tridentate carboxylic acid that does not incorporate further functional groups or is a tridentate carboxylic acid that incorporates
15 further functional groups (e.g. from 1 to 10, such as 1 to 5, such as 1 to 3 further functional groups), optionally wherein the organic ligand is selected from one or more of benzene-1,3,5-tribenzoate, 5′-(4-carboxyphenyl)-2′-methyl[1,1′:3′,1″-terphenyl]-4,4″-dicarboxylic acid, 2′-amino-5′-(4-carboxyphenyl)-[1,1′:3′,1″-terphenyl]-4,4″-dicarboxylic acid, 4,4′,4″-(1,3,5-triazine-2,4,6-triyl)tribenzoic acid, and 4,4′,4″-(pyridine-2,4,6-triyl)tribenzoic acid.
16. The suspension according to any one of Clause 9, or Clauses 12 to 15, the sol according to any one of Clause 10 or Clauses 12 to 15, or the composition according to any one of Clauses 11 to 15, wherein, when present, the capping molecule is a monodentate carboxylic acid, optionally wherein the capping ligand is selected from one or more of acetic acid, formic acid, and other monodentate acids, for example the capping molecule is selected from acetic acid and/or formic acid (e.g. the capping molecule is formic acid).
17. The suspension according to any one of Clause 9, or Clauses 12 to 16, the sol according to any one of Clause 10 or Clauses 12 to 16, or the composition according to any one of Clauses 11 to 16, wherein the MOF is NUS-8 or an analogue thereof having:
(a) Zr⁴⁺ as the metal ion, benzene-1,3,5-tribenzoate as the organic ligand, and formic acid as the capping molecule;
(b) Zr⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(c) Zr⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(d) Hf⁴⁺ as the metal ion, benzene-1,3,5-tribenzoate as the organic ligand, and formic acid as the capping molecule;
(e) Hf⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(f) Hf⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(g) Zr⁴⁺ as the metal ion and benzene-1,3,5-tribenzoate as the organic ligand;
(h) Zr⁴⁺ as the metal ion and 5′-(4-carboxyphenyl)-2′-methyl[1,1′:3′,1″-terphenyl]-4,4″-dicarboxylicacid as the organic ligand;
(i) Zr⁴⁺ as the metal ion and 2′-amino-5′-(4-carboxyphenyl)-[1,1′:3′,1″-terphenyl]-4,4″-dicarboxylic acid as the organic ligand;
(j) Hf⁴⁺ as the metal ion and benzene-1,3,5-tribenzoate as the organic ligand;
(k) Hf⁴⁺ as the metal ion and 5′-(4-carboxyphenyl)-2′-methyl[1,1′:3′,1″-terphenyl]-4,4″-dicarboxylicacid as the organic ligand; or
(l) Hf⁴⁺ as the metal ion and 2′-amino-5′-(4-carboxyphenyl)-[1,1′:3′,1″-terphenyl]-4,4″-dicarboxylic acid as the organic ligand.
18. The suspension according to any one of Clause 9, or Clauses 12 to 17, the sol according to any one of Clause 10 or Clauses 12 to 17, or the composition according to any one of Clauses 11 to 17, wherein the MOF is NUS-8 or an analogue thereof having:
(a) Zr⁴⁺ as the metal ion, benzene-1,3,5-tribenzoate as the organic ligand, and formic acid as the capping molecule;
(b) Zr⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule; or
(c) Zr⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(d) Zr⁴⁺ as the metal ion and benzene-1,3,5-tribenzoate as the organic ligand;
(e) Zr⁴⁺ as the metal ion and 5′-(4-carboxyphenyl)-2′-methyl[1,1′:3′,1″-terphenyl]-4,4″-dicarboxylicacid as the organic ligand; or
(f) Zr⁴′ as the metal ion and 2′-amino-5′-(4-carboxyphenyl)-[1,1′:3′,1″-terphenyl]-4,4″-dicarboxylic acid as the organic ligand.
19. The suspension according to any one of Clause 9, or Clauses 12 to 19, the sol according to any one of Clause 10 or Clauses 12 to 19, or the composition according to any one of Clauses 11 to 19, wherein the MOF has a thickness of from 8 to 20 nm, such as from 10 to 15 nm, such as about 13 nm.
20. The composition according to any one of Clauses 11 to 19, wherein the composition is in the form of a gel (e.g. an aerogel or a xerogel) or a film.
21. The composition according to Clause 20, wherein the film has one or more of the following properties:
(a) a thickness of from 50 to 1000 nm, such as from 80 to 850 nm;
(b) a Young's modulus of from 1.4 to 2.5 GPa, such as from 1.5 to 2 GPa, such as about 1.6 GPa;
(c) an adhesion energy of from 200 to 500 E⁻¹⁸ J, such as from 300 to 400 E⁻¹⁸ J, such as about 340 E⁻¹⁸ J;
(d) the film is capable of being folded from 100 to 1000 times, such as from 250 to 500 times, such as about 200 times without degradation of its physical properties.
22. A method of making a gel from a suspension as described in any one of Clause 9, or Clauses 12 to 19, wherein the method comprises the steps of
(a) providing the suspension as described in any one of Clause 9, or Clauses 12 to 19; and
(b) removing the water and/or THF to evaporate to form a gel.
23. The method according to Clause 22, wherein the removal of water and/or THF is accomplished using one or more of evaporation of room temperature, evaporation under vacuum at room temperature and/or at elevated temperature, freeze drying, and supercritical carbon dioxide drying.
24. The method according to Clause 23, wherein when the method applied is selected from evaporation of room temperature, evaporation under vacuum at room temperature and/or at elevated temperature, then the gel is obtained in the form of a xerogel.
25. The method according to Clause 23, wherein when the method applied is selected from freeze drying or supercritical carbon dioxide drying, then the gel is obtained in the form of an aerogel.
26. A method of making a sol as described in any one of Clause 10, or Clauses 12 to 19, wherein the method comprises the steps of
(a) providing a gel as described in Clause 20, as dependent upon any one of Clauses 11 to 19; and
(b) adding a solvent to the gel to form the sol.
27. The method according to Clause 26, wherein the gel is obtained by the method according to any one of Clauses 22 to 26.

### DRAWINGS

FIG. 1 depicts the schematic representation for the direct and scalable synthesis of homogeneous and stable suspensions composed of ultra-large metal-organic nanosheets (MONs) with excellent solution processability. (a) Synthesis strategy; (b) Thermodynamic control on the synthesis based on molecular dynamics (MD) simulations. The dotted rectangle compares the interaction between dimethylformamide molecules and NUS-8 nanosheet with that of NUS-8 interlayers; (c) Growth kinetics control on the formation of MONs; and (d) Demonstrations on the solution processability of the obtained suspensions.

FIG. 2 depicts the simulation models for calculating the interactions between formic acid/4,4′,4″,-benzene-1,3,5-triyl-tris(benzoate) (BTB) linker and the metal clusters in different positions: (a) Position A and B of the Zr-oxo clusters; (b) BTB and position A; (c) Formic acid and position A; and (d) Formic acid and position B.

FIG. 3 depicts the optimized interaction pairs reflected by MD simulations: (a) H₂O/NUS-8 nanosheet; (b) Acetone/NUS-8; (c) NUS-8 interlayer interaction; (d) H₂O/NUS-8-NH₂ nanosheet; (e) N,N-dimethylformamide (DMF)/NUS-8-NH₂; (f) Acetone/NUS-8-NH₂; and (g) NUS-8-NH₂ interlayer interaction. The right column lists the corresponding adsorption energies of the interaction pairs.

FIG. 4 depicts (a,b) optical photographs of the as-synthesized NUS-8 sols composed of unexceptionally large nanosheets and after shaking indicative of the Tyndall effect; (c) reversible NUS-8 sol-gel transformation upon adding or removing solvents such as DMF, tetrahydrofuan (THF), or H₂O; (d) scanning electron microscopy (SEM) image of one-piece 30 NUS-8 nanosheet deposited on the silicon (Si) surface; (e) atomic force microscopy (AFM) image of NUS-8 nanosheet with height profile; (f) low-magnification transmission electron microscopy (TEM) image of NUS-8 nanosheet; (g,h) high angle annular dark field scanning transmission electron microscopy (HAADF-STEM) images of NUS-8 nanosheet. The inset in (g) presents the intensity line profile of the lattice as indicated by the dotted pink line. The inset 35 in (h) depicts the schematic illustration of the NUS-8 framework viewed from c-direction; and (i,j) X-ray diffractometer (XRD) patterns and N₂ sorption isotherms under 77 K of NUS-8 powder with different functional groups. The theoretical XRD pattern of NUS-8 powder is also given for comparison.

FIG. 5 depicts the effect of different solvents and capping molecule on the solvothermal formations of NUS-8 suspensions: (a) Without addition of H₂O; (b) Substitution of H₂O by equivalent absolute ethanol; (c) Substitution of H₂O by equivalent acetone; and (d) Substitution of formic acid by equivalent acetic acid. The pristine composition of precursor solution: 15 mL DMF+2 mL H₂O+2 mL formic acid, m(ZrCl₄)=30 mg, m(H₃BTB)=30 mg.

FIG. 6 depicts the optical photographs of the synthesis of NUS-8 sol in a large container (a-b) and NUS-8 gels after different treatments (c-f). (c) Redispersion of “non-flowing” gel in H₂O after three days' storage at ambient condition. The gel was primarily dispersed in H₂O, and the container was left uncapped; (d) The gel as shown in (c) after heating at 60° C. for 2 h; (e) The gel as shown in (d) after heating at 80° C. for 2 h; and (f) Non-dispersible dried gel in H₂O. Heating the bottle containing the dried gel at 80° C. for 2 h witnessed nearly identical look.

FIG. 7 depicts the dispersions of NUS-8 gels within various solvents DEF: diethylformide; and DMA: dimethylacetamide.

FIG. 8 depicts more SEM images of monodispersed NUS-8 nanosheets on Si wafer.

FIG. 9 depicts additional AFM images of monodispersed NUS-8 nanosheets on Si surface. The second row presents the height profiles of (a-d), respectively. Note that imaging on more NUS-8 nanosheets with larger areas is not feasible due to the limitation of the AFM scanner. In such a fashion, only partial NUS-8 nanosheets have been imaged.

FIG. 10 depicts (a) optical photograph of the as-synthesized NUS-8(Zr) nanosheets under short growth periods; (b) optical photograph of the as-synthesized NUS-8(Zr) nanosheets after 1 h growth; (c, d) AFM images of the nanosheets deposited onto Si surface; and (e) height profiles of the positions as indicated in (c).

FIG. 11 depicts (a-c) AFM images of NUS-8(Zr) nanosheets deposited on Si surface. The nanosheets were obtained after 5 h growth; and (d) height profiles of the positions as indicated in (a).

FIG. 12 depicts STEM images of NUS-8(Zr) nanosheets obtained after 1 h growth.

FIG. 13 depicts the Fourier-transformed extended X-ray absorption fine structure (EXAFS) spectra of the NUS-8(hafnium (Hf)) powder and sol. Note: The EXAFS spectra of NUS-8(Hf) powder and sol register identical fingerprints, suggesting identical chemical environments of the Hf centers within NUS-8 frameworks under both dry and wet forms.

FIG. 14 depicts the attenuated total reflectance-Fourier transform infrared (ATR-FTIR) spectra of the “non-flowing” NUS-8 gel under intermittent heating, in comparison with that of its dry form.

FIG. 15 depicts the XRD pattern of NUS-8(Hf) nanosheets in comparison with the simulated one. The inset presents the optical photograph of the as-synthesized NUS-8(Hf) nanosheets.

FIG. 16 depicts the Fourier-transformed EXAFS spectrum of NUS-8(Hf) nanosheets. Note: NUS-8(Hf) can be detected by our facility, which failed to detect Zr K-edge.

FIG. 17 depicts the ATR-FTIR spectra of the used organic linkers.

FIG. 18 depicts (a) optical photograph of the scalable synthesis of NUS-8-CH₃ nanosheets; and (b-j) SEM images of monodispersed NUS-8-CH₃ nanosheets on Si wafer.

FIG. 19 depicts the AFM images of monodispersed NUS-8-CH₃ nanosheets on Si surface. The second row presents the height profiles of (a-d), respectively.

FIG. 20 depicts (a) optical photograph of the scalable synthesis of NUS-8-NH₂ nanosheets; 25 and (b-j) SEM images of monodispersed NUS-8-NH₂ nanosheets on Si wafer.

FIG. 21 depicts the AFM images of monodispersed NUS-8-NH₂ nanosheets on Si surface. The second column presents the height profiles of (a, c), respectively.

FIG. 22 depicts the ATR-FTIR spectra of NUS-8 powders without and with different functional groups.

FIG. 23 depicts the thermogravimetric analysis (TGA) curves of NUS-8 powders without and with different functional groups.

FIG. 24 depicts (a) optical photograph of NUS-8 monolith; (b) schematic representation of the formation of NUS-8 films by drop-casting; (c) representative top-view SEM image of NUS-8 films; (d-h) cross-sectional SEM images of a series of NUS-8 films on Si substrates with tunable thickness. Solid concentration and solution volume used for films: (d) C_{NUS-8}=0.17 g·L⁻¹, 10 μL; (e) C_{NUS-8}=0.5 g·L⁻¹, 10 μL; (f) C_{NUS-8}=1 g·L⁻¹, 10 μL; (g) C_{NUS-8}=1 g·L⁻¹, 20 μL; and (h) C_{NUS-8}=1 g·L⁻¹, 30 μL; (i) optical photograph of the large-area NUS-8 film on polystyrene (PS) substrate fabricated by doctor-blading. C_{NUS-8}=3.2 g·L⁻¹, volume=8 mL; and (j,k) cross-sectional SEM images of the film as shown in (i).

FIG. 25 depicts the zoomed-in SEM images of NUS-8 monolith.

FIG. 26 depicts the N₂ sorption isotherms under 77 K of NUS-8 xerogel dried under vacuum at 25° C. Note: It is different from the ones shown in FIG. 4j that are typically dried under ambient conditions.

FIG. 27 depicts the pore size distribution of NUS-8 xerogel as shown in FIG. 26, dried under vacuum at 25° C.

FIG. 28 depicts the CO₂ sorption isotherms at 298 K of the NUS-8 xerogel dried under vacuum at 25° C.

FIG. 29 depicts the N₂ sorption isotherms under 77 K of NUS-8 aerogel fabricated by freezing-drying.

FIG. 30 depicts the pore size distribution of NUS-8 aerogel as shown in FIG. 29, dried by freezing-drying.

FIG. 31 depicts the CO₂ sorption isotherms at 298 K of the NUS-8 aerogel obtained by freezing-drying.

FIG. 32 depicts the folding tests toward NUS-8 films on flexible PS substrate: (a) The schematic representation of the test; (b1, b2) AFM images of pristine NUS-8 film on PS; (c1, c2) AFM images of the NUS-8 film on PS after 100 times' folding. Imaging was conducted nearby the boundary as indicated in (a); and (d1, d2) AFM images of the NUS-8 film on PS after 200 times' folding. Imaging was also conducted nearby the boundary as indicated in (a).

FIG. 33 depicts the top-view SEM images of NUS-8 films as presented respectively in FIG. 24e-h.

FIG. 34 depicts (a) optical photograph of the 8-inch wafer-scale fabrication of high-quality NUS-8 thin film by doctor-blading. C_{NUS-8}=3.3 g-LA. The arrows indicate the edge of the film; (b) grazing-incidence wide-angle X-ray scattering (GIWAXS) out-of-plane and in-plane scattering profiles of NUS-8 thin film in comparison with its powder form; (c) two-dimensional (2D)-GIWAXS image of the as-fabricated NUS-8 thin film; and (d) representative force-displacement curves collected between a diamond tip and NUS-8 film. The inset highlights the approach and retraction of the tip during the nanomechanical measurement. Note that the films used for GIWAXS and nanoindentation measurements all have a thickness of 390 nm; the thickness of the film, as shown in (a), is 400 nm (see FIG. 36).

FIG. 35 depicts the SEM images of NUS-8 film (a, b) before; and (c-f) after multiple scotch tape tests.

FIG. 36 depicts the cross-sectional SEM images of NUS-8 film fabricated on an 8-inch Si wafer.

### DESCRIPTION

It has been surprisingly found that it is possible to directly form 2D MOF suspensions possessing permanent porosity and excellent fluidity. In addition, the excellent solution processability of the MOF suspensions enables the formation of MOF monolith, aero- and xerogels with adjustable mesoporosity, as well as large-area textured films with great homogeneity, controllable thickness and appreciable mechanical properties.

Thus in a first aspect of the invention, there is provided a method of forming a metal-organic framework (MOF) suspension, the process comprising:
(a) providing a metal precursor, an organic ligand, a capping molecule and a solvent comprising a polar aprotic solvent and water;
(b) adding the metal precursor, organic ligand and capping molecule to the solvent to provide a reaction mixture and heating it for a period of time to form the MOF suspension, wherein:
   the metal precursor has a concentration in the solvent of from 1.0 to 2 mg/mL;
   the organic ligand has a concentration in the solvent of from 1.0 to 2 mg/mL;
   the capping molecule is provided in an amount of from 5 to 20 vol % relative to the total volume of the solvent, and/or it is provided in an amount of from 5 to 20 wt % relative to the total weight of the solvent; and
   water represents from 5 to 20 vol % of the solvent, with the balance being the polar aprotic organic solvent.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “a metal precursor” includes mixtures of two or more such metal precursors, reference to “a solvent” includes mixtures of two or more such solvents, and the like.

The term “metal precursor” when used herein means any metal compound that comprises a metal suitable for forming a MOF. Examples of suitable metals include, but are not limited to Zr, Hf, Al, Cr, Fe and Ti. Examples of suitable metal precursors include, but are not limited to, ZrOX₂, HfOX₂, ZrX₄, HfX₄, AlX, Crk, Feh, TiX₄, and combinations thereof, where X is halo selected from Cl, Br, I, or F. In particular embodiments that may be mentioned herein, the metal precursor may be selected from one or more of ZrX₄ and HfX₄. In further embodiments that may be mentioned herein, the metal precursor may be ZrCl₄ or HfCl₄.

As noted above, the concentration of the metal precursor in the solvent may be any concentration of from 1.0 to 2 mg/mL. Examples of particular concentrations for the metal precursor in the solvent may be from 1.5 to 1.8 mg/mL or about 1.76 mg/mL.

The term “organic ligand” in the context of the current invention will be understood to mean an organic compound that can be used to form the main organic component of a MOF, in particular it may refer to an organic compound that occupies coordination sites around the metal ions of the MOF in the 2D plane. For example, the organic ligand may be a tridentate carboxylic acid that does not incorporate further functional groups or it may be a tridentate carboxylic acid that incorporates further functional groups (e.g. from 1 to 10, such as from 1 to 5, such as from 1 to 3 further functional groups). Examples of suitable functional groups include, but are not limited to C₁₋₆ alkyl, amino, hydroxyl, OC_{I}.e alkyl, thiol, and SC₁₋₆ alkyl. The term “amino” when used herein may refer to an unsubstituted NH₂ group or it may refer to a group where one or both of the hydrogen atoms are replaced by a C₁₋₆ alkyl group. Examples of organic ligands that may be mentioned herein include, but are not limited to one or more of benzene-1,3,5-tribenzoate, 5′-(4-carboxyphenyl)-2′-methyl[1,1′:3′,1″-terphenyl]-4,4″-dicarboxylic acid, 2′-amino-5′-(4-carboxyphenyl)-[1,1′:3′,1″-terphenyl]-4,4″-dicarboxylic acid, 4,4′,4″-(1,3,5-triazine-2,4,6-triyl)tribenzoic acid, and 4,4′,4″-(pyridine-2,4,6-triyl)tribenzoic acid.

Unless otherwise stated, the term “alkyl” refers to an unbranched or branched, acyclic or cyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₆ cycloalkyl. In particular embodiments of the invention, may be unsubstituted. In additional or alternative embodiments of the invention, alkyl may be methyl.

As noted above, the concentration of the organic ligand in the solvent may be from 1.0 to 2 mg/mL. In particular embodiments of the invention, the concentration of the organic ligand in the solvent may be from 1.5 to 1.8 mg/mL, such as about 1.76 mg/mL.

The concentration of the organic ligand and the metal precursor in the solvent may be different or, more particularly, the same.

When used herein, the term “capping molecule” refers to an organic compound capable of occupying coordination sites that lie outside the 2D plane of the MOF. For example, the organic compound may be a monodentate ligand, such as a monodentate carboxylic acid. Examples of capping molecules that may be mentioned herein include, but are not limited to acetic acid, formic acid, other monodentate acids, and combinations thereof. In particular embodiments of the invention, the capping molecule may be acetic acid or formic acid, or both. In more particular embodiments of the invention, the capping molecule may be formic acid. Without wishing to be bound by theory, it is believed that the use of these capping molecules may enable the generation of large-area MOFs with good solution processability.

As noted above, the capping molecule may be provided in an amount of from 5 to 20 vol % relative to the total volume of the solvent, and/or may be provided in an amount of from 5 to 20 wt % relative to the total weight of the solvent. For example, the capping molecule may be provided in an amount of from 10 to 15 vol % relative to the total volume of the solvent, and/or it may be provided in an amount of from 10 to 15 wt % relative to the total weight of the solvent. In particular embodiments of the invention, the capping molecule may be provided in an amount of about 11.8 vol % relative to the total volume of the solvent, and/or may be provided in an amount of about 15 wt % relative to the total weight of the solvent.

The methods disclosed herein involve washing steps that may, or in some cases may not, remove the capping molecule. As such, the capping molecule may or not be present in the MOF.

When used herein, the term “solvent” refers to a solvent system that includes a polar aprotic solvent and water. For example, water may be provided in an amount of from 5 to 20 vol % of the solvent, with the balance being the polar aprotic solvent. Examples of suitable polar aprotic solvent include, but are not limited to N,N-dimethylformamide (DMF), acetone, acetonitrile, dimethyl sulfoxide, dimethylacetamide, N,N-diethylformamide, and combinations thereof. In particular embodiments that may be mentioned herein, the polar aprotic solvent may be selected from one or more of N,N-dimethylformamide, dimethylacetamide, and N,N-diethylformamide. In more particular embodiments that may be mentioned herein, the polar aprotic solvent may be N,N-dimethylformamide.

Any suitable period of time may be used in step (b) of the process disclosed above. For example, the period of time may be from from 1 hour to 72 hours, such as about 24 hours.

The heating applied to step (b) of the process above may be any suitable temperature. For example, the heating applied to the reaction mixture of step (b) may be from 100 to 150° C., such as about 120° C.

The MOF suspension obtained in the first aspect of the invention may be purified. This may be achieved by the following additional steps:
(i) removing the MOF from the reaction mixture by centrifugation;
(ii) adding a polar aprotic solvent to the removed MOF to form a suspension and then removing the MOF by centrifugation;
(iii) repeating step (b) from one to ten times; and
(iv) adding water to the removed MOF to form a suspension and then removing the MOF by centrifugation and repeating from two to ten times (e.g. from 4 to 6 times, such as five times) over a 24-hour period;
(v) adding water and/or tetrahydrofuran to the removed MOF to provide a final suspension of the MOF.

As will be appreciated, it is believed that the MOF suspensions disclosed herein can be either used as liquid adsorbents or processed as mixed matrix membranes with relatively high loading or just as pure MOF membranes with good mechanical properties and salient separation performance.

Thus, in a second aspect of the invention, there is provided an aqueous and/or tetrahydrofuran suspension (e.g. a colloidal suspension) of a MOF comprising:
water and/or tetrahydrofuran (THF); and
MOF nanosheets having an area of greater than or equal to 10,000 μm².

In a third aspect of the invention, there is provided a sol comprising: a solvent (e.g. selected from one or more of water, THF and DMF); and MOF nanosheets having an area of greater than or equal to 10,000 μm².

The sols disclosed herein may remain stable for an extended period of time. For example, the sols may be stable for a period of two years or even more. For example, a sol described in the examples of the current application has been stable for a period of 2.5 years at the date that the provisional application for this patent application was filed.

The methods disclosed herein may be useful in the manufacture of MOF nanosheets. Thus, in a fourth aspect of the invention, there is provided a composition comprising MOF nanosheets having an area of greater than or equal to 10,000 μm².

The MOF nanosheets disclosed herein may have any suitable area greater than or equal to 10,000 μm². For example, the MOF nanosheets may have an area of from 10,000 to 30,000 μm². In particular embodiments that may be mentioned herein, the MOF nanosheets may have an area of from 15,000 to 29,000 μm². In more particular embodiments that may be mentioned herein, the MOF nanosheets may have an area of from 18,000 to 25,000 μm².

For the avoidance of doubt, reference to MOF nanosheets herein may refer to the MOF nanosheets per se or as part of a composition; an aqueous and/or tetrahydrofuran suspension; or a sol.

The MOF in the MOF nanosheets disclosed herein may have any suitable thickness. For example, the MOF may have a thickness of from 8 to 20 nm. In particular embodiments that may be mentioned herein, the MOF in the MOF nanosheets may have a thickness of from 10 to 15 nm. In more particular embodiments that may be mentioned herein, the MOF in the MOF nanosheets may have a thickness of 13 nm.

The MOF in the MOF nanosheets disclosed herein may be NUS-8 and/or an analogue thereof, wherein the MOF is formed from:
(a) a metal ion and an organic ligand; or
(b) a metal ion and an organic ligand and a capping molecule.

When used herein, NUS-8 specifically refers to a MOF comprising ZreO₄(OH)₄ or Hf₆ O₄(OH)₄ clusters (i.e. Zr⁴⁺ or Hf⁴⁺ as the metal ion) and 1,3,5-benzenetribenzoate (BTB) as the organic ligand. Analogues of NUS-8 may refer to the replacement of the organic ligand with a different ligand (e.g. a BTB ligand incorporating one or more substituents, for example selected from C₁₋₆ alkyl, amino, hydroxyl, OC₁.a alkyl, thiol, and SC₁.a alkyl). As will be appreciated any of the ligands mentioned above in relation to the method may be used in the NUS-8 analogues. Additionally, or alternatively analogues of NUS-8 may be obtained by replacing the metal ion cluster with a different metal (e.g. Al³⁺, Cr³⁺, Fe³⁺, and Ti⁴⁺). As will be appreciated, the methods disclosed herein may be particularly suited to the manufacture of NUS-8 or analogues thereof.

For the avoidance of doubt, the metal ion, organic ligand, and capping molecule described above are as described hereinbefore and have the same properties. As such, for brevity, these materials that have been discussed will not be discussed again here.

The MOF nanosheets disclosed herein may be selected from NUS-8 or an analogue thereof having:
(a) Zr⁴⁺ as the metal ion, benzene-1,3,5-tribenzoate as the organic ligand, and formic acid as the capping molecule;
(b) Zr⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(c) Zr⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(d) Hf⁴⁺ as the metal ion, benzene-1,3,5-tribenzoate as the organic ligand, and formic acid as the capping molecule;
(e) Hf⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(f) Hf⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(g) Zr⁴⁺ as the metal ion and benzene-1,3,5-tribenzoate as the organic ligand;
(h) Zr⁴⁺ as the metal ion and 5′-(4-carboxyphenyl)-2′-methyl[1,1′:3′,1″-terphenyl]-4,4″-dicarboxylicacid as the organic ligand;
(i) Zr⁴⁺ as the metal ion and 2′-amino-5′-(4-carboxyphenyl)-[1,1′:3′,1″-terphenyl]-4,4″-dicarboxylic acid as the organic ligand;
(j) Hf⁴⁺ as the metal ion and benzene-1,3,5-tribenzoate as the organic ligand;
(k) Hf⁴⁺ as the metal ion and 5′-(4-carboxyphenyl)-2′-methyl[1,1′:3′,1″-terphenyl]-4,4″-dicarboxylicacid as the organic ligand; and
(l) Hf⁴⁺ as the metal ion and 2′-amino-5′-(4-carboxyphenyl)-[1,1′:3′,1″-terphenyl]-4,4″-dicarboxylic acid as the organic ligand.

More particularly, the MOF nanosheets may be formed from NUS-8 or an analogue thereof having:
(a) Zr⁴⁺ as the metal ion, benzene-1,3,5-tribenzoate as the organic ligand, and formic acid as the capping molecule;
(b) Zr⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule; or
(c) Zr⁴′ as the metal ion, 2′-amino-5′-(4-carboxyphenyl)-[1,1′:3′,1″-terphenyl]-4,4″-dicarboxylic acid as the organic ligand, and formic acid as the capping molecule;
(d) Zr⁴⁺ as the metal ion and benzene-1,3,5-tribenzoate as the organic ligand;
(e) Zr⁴⁺ as the metal ion and 5′-(4-carboxyphenyl)-2′-methyl[1,1′:3′,1″-terphenyl]-4,4″-dicarboxylicacid as the organic ligand; or
(f) Zr⁴⁺ as the metal ion and 2′-amino-5′-(4-carboxyphenyl)-[1,1′:3′,1″-terphenyl]-4,4″-dicarboxylic acid as the organic ligand.

As will be appreciated, the second, third and fourth aspects of the invention may be provided in the form of a gel or a film. For example, the second, third and fourth aspects of the invention may be provided in the form of an aerogel or a xerogel.

The gel disclosed herein may be prepared from a suspension by:
(a) providing the suspension provided by the second aspect of the invention; and
(b) removing the water and/or THF to evaporate to form a gel.

Examples of suitable methods to remove water and/or THF include, but are not limited to one or more of evaporation of room temperature, evaporation under vacuum at room temperature and/or at elevated temperature, freeze drying, supercritical carbon dioxide drying, and technically sensible combinations thereof. For example, the removal of water and/or THF is accomplished using one or more of evaporation of room temperature, evaporation under vacuum at room temperature and/or at elevated temperature, freeze drying, and supercritical carbon dioxide drying.

As will be appreciated, when the method applied is selected from evaporation of room temperature, evaporation under vacuum at room temperature and/or at elevated temperature, then the gel is obtained in the form of a xerogel. When the method applied is selected from freeze drying or supercritical carbon dioxide drying, then the gel is obtained in the form of an aerogel.

The sol disclosed herein may be prepared by:
(a) providing a gel described hereinbefore; and
(b) adding a solvent to the gel to form the sol.

Also disclosed herein is a gel obtained or obtainable by the processes to form a gel mentioned hereinbefore.

The film disclosed herein may have one or more of the following properties (e.g. see Example 7):
(a) a thickness of from 50 to 1000 nm, such as from 80 to 850 nm;
(b) a Young's modulus of from 1.4 to 2.5 GPa, such as from 1.5 to 2 GPa, such as about 1.6 GPa;
(c) an adhesion energy of from 200 to 500 E⁻¹⁸ J, such as from 300 to 400 E⁻¹⁸ J, such as about 340 E⁻¹⁸ J; and
(d) the film is capable of being folded from 100 to 1000 times, such as from 250 to 500 times, such as about 200 times without degradation of its physical properties.

It will be appreciated that the invention provides a facile fabrication of films with great 35 homogeneity, controllable thickness and appreciable mechanical properties. In addition, as described in Example 7, the successful integration of MONs on electronic devices was achieved.

Taking these advantages together, the invention holds great promise in the integration and mass-production of MOFs in miniaturized electronics devices and membrane-based separation technologies. In addition, the invention can be extended to other porous frameworks to address some technical challenges existing in porous materials, electronics, and optoelectronics.

Further aspects and embodiments of the invention will now be described by reference to the following non-limiting examples.

### EXAMPLES

### Materials

The chemicals for the synthesis of MOF suspensions were purchased from commercial suppliers: zirconium(IV) chloride (ZrCl₄, >99%, Materion), formic acid (>99%, TCI), 4,4′,4″,-benzene-1,3,5-triyl-tris(benzoic acid) (H₃BTB, >99%, Yanshen Technology Co., Ltd.), 5′-(4-carboxyphenyl)-2′-methyl[1,1′:3′,1″-terphenyl]-4,4″-dicarboxylic acid (H₃BTB-CH₃, >98%, Yanshen Technology Co., Ltd.), 2′-amino-5′-(4-carboxyphenyl)-[1,1′:3′,1″-terphenyl]-4,4″-dicarboxylic acid (H₃BTB-NH₂, >98%, Yanshen Technology Co., Ltd.), DMF (>99.8%, Sigma-Aldrich), hafnium(IV) chloride (>98%, Sigma-Aldrich), tetrahydrofuran (>99.9%, Sigma-Aldrich), absolute ethanol (>99.5%, Sigma-Aldrich), Si substrate (>99%, Pioneer Sci-Tech Pte. Ltd.), and polystyrene sheets (GregTech). All the chemicals were used without further purification.

### Analytical Techniques

### SEM Imaging

SEM was conducted via a JEOL JSM-7610F.

### XRD

XRD was conducted via a XRD (Bruker) equipped with Cu Kα radiation (λ=1.5406 Å) under a Bragg-Brentano geometry with a step size of 0.02° and a scan speed of 0.5°·min⁻¹.

### TEM Imaging

TEM imaging was conducted via a JEOL TEM (JEM-3010).

### HAADF-STEM Imaging

HAADF-STEM imaging was performed by a JEM-ARM200F (JEOL) microscope equipped with an ASCOR aberration corrector and a cold-field emission gun, operated at 200 kV.

### N₂ Sorption Isotherms

N₂ sorption isotherms were collected under 77 K using a Micromeritics ASAP 2020 surface area and pore size analyser.

### TGA

TGA was performed using a Shimadzu DTG-60AH up to 800° C. under an N₂ flowing rate of 50 mL·min⁻¹ and a heating-up speed of 5° C.·min⁻¹.

### 2D GIWAXS

2D GIWAXS measurements were performed on Nanoinxider (Xenocs) with Cu Kα radiation as the source at an incidence angle of 0.2°.

### Hf K-Edge Spectroscopy

The Hf K-edge spectra were processed according to the conventional procedure using the IFEFFIT package.

### NMR Spectroscopy

NMR spectroscopy was performed on BrukerAvance 400 MHz NMR spectrometer (DRX400).

### Example 1. Synthesis of NUS-8 Suspensions with Variable Functionalities

### NUS-8 Suspensions

The synthesis of NUS-8 suspensions was conducted at 120° C. for 24 h in mixture solutions containing ZrCl₄ (30 mg) and H₃BTB (30 mg). The solution was composed of DMF (15 mL), ultrapure water (2 mL), and formic acid (2 mL). The resulting viscous suspensions were rinsed with fresh DMF five times using a centrifuge at a speed of 3500 rpm and then soaked in fresh DMF overnight. Afterward, the suspensions were soaked in fresh ultrapure water and absolute ethanol for another two days while renewing the solvent five times per day.

### NUS-8-CH₃ Suspensions

NUS-8-CH₃ suspensions were prepared from ZrCl₄ (30 mg) and H₃BTB-CH₃ (30 mg) by following the protocol above.

### NUS-8-NH₂ Suspensions

NUS-8-NH₂ suspensions were prepared from ZrCl₄ (30 mg) and H₃BTB-NH₂ (30 mg) by following the protocol above.

### Results and Discussion

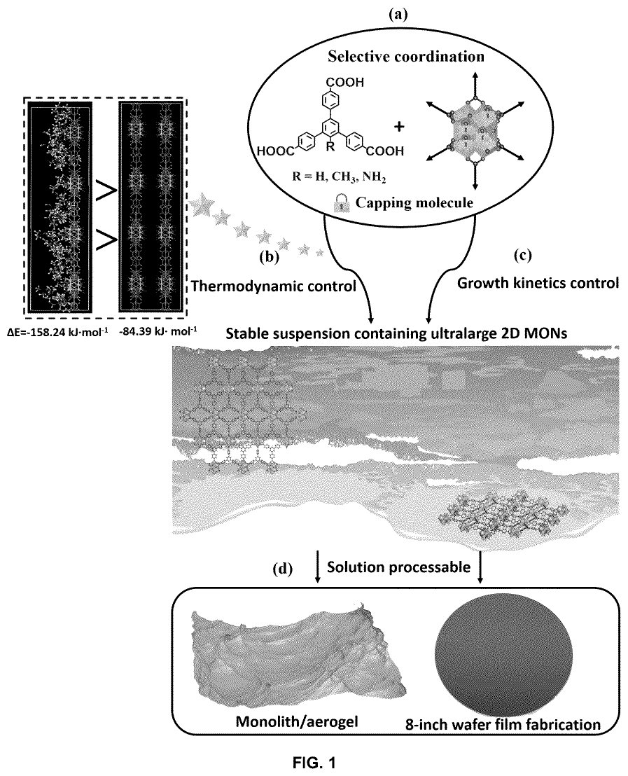

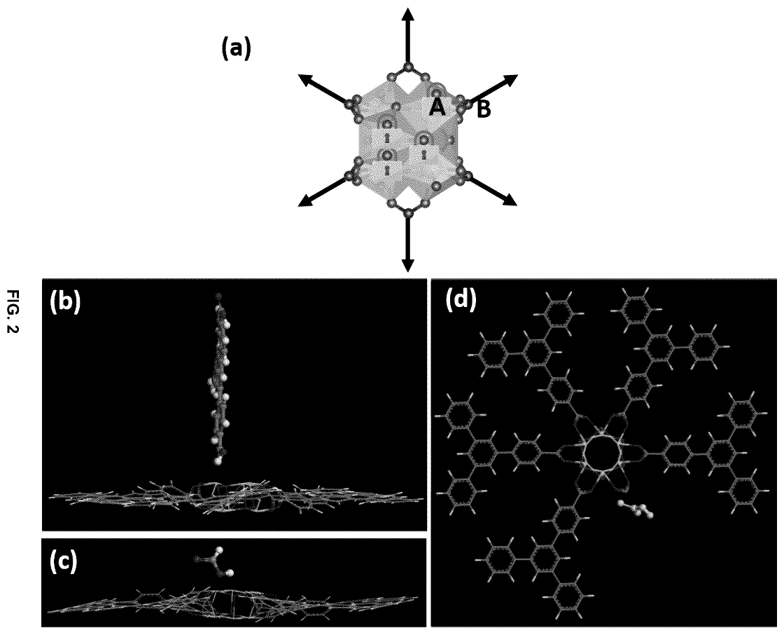

A one-pot, surfactant-free, and scalable synthesis of highly stable MOF suspensions composed of exceptionally large (average area>15000 μm²) NUS-8 nanosheets with variable functionalities and excellent solution processability is reported. This is achieved by three main considerations. Firstly, the chosen 2D MOF (named NUS-8) with kagome dual topology, built alternatively by 6-connected Zre SBUs and trigonal carboxylate linker BTB moieties, can also form an interpenetrated 3D framework under similar synthetic conditions (R. Wang et al., Inorg. Chem. 2014, 53, 7086; Z. Hu et al., J. Mater. Chem. A 2017, 5, 8954; and Z.-R. Tao et al., Nat. Commun. 2019, 10, 2911). A selective coordination strategy was adopted to realize the scalable synthesis of large and multilayered NUS-8 nanosheets with various functional groups (—H, —CH₃, —NH₂). Formic acid as the capping agent was added during the synthesis to selectively occupy six coordination sites of the clusters out of the 2D plane and to leave the rest six sites within the 2D plane unoccupied (FIG. 1a). The selective coordination of formic acid with metal cluster sites out of the 2D plane probably stems from the energetically favorable adsorption of formic acid and thus coordination with those sites of metal clusters (FIG. 2, Table 1). As indicated in FIG. 2 and Table 1, interactions between formic acid and different positions of the Zr-oxo clusters are different, whereas the interaction energy value between formic acid and position B is −8.33 kJ·mol⁻¹, much smaller than that (−14.87 kJ·mol⁻¹) between formic acid and position A. This suggests that formic acid is energetically favorable to be adsorbed by the A positions within the Zr-oxo dusters and further became coordinated with those sites. Meanwhile, interactions between BTB linkers and the Zr-oxo clusters in A position are weaker compared with that between formic acid and A positions of the Zr-oxo clusters (Table 1). Additionally, the BTB linkers prefer to bind with the Zr-oxo clusters in B position other than those in A position to reduce the steric hindrance. This will undoubtedly minimize the system energy to a large extent and lead to large NUS-8 nanosheets.

Further coordination between the unoccupied sites of the clusters and BTB linkers afforded NUS-8 nanosheets with less steric hindrance and more stability. Secondly, distinct from previous studies that typically rely on supersaturated precursor solutions for the progressive formation of colloidal suspension composed of nanosized MOF crystallites (A. K. Chaudhari, I. Han & J.-C. Tan, Adv. Mater. 2015, 27, 4438; B. Bueken et al., Chem. Sci. 2017, 8, 3939; and J. Hou, A. F. Sapnik & T. D. Bennett, Chem. Sci. 2020, 11, 310), we utilized a diluted reactant solution to limit the rapid and excessive nucleation of NUS-8 that will dramatically consume the reactant. As a result, a slow and equilibrium-based growth of large NUS-8 nanosheets can be reasonably envisioned. Thirdly, interlayer interactions of MOFs need to be compensated to avoid agglomeration. Increasing MOF nanosheets-solvent interactions via judicious choices of solvent indeed stabilizes the resulting nanosheets (FIG. 1b), and good solution processability can thus be harvested.

TABLE 1
Interaction energies between formic acid/BTB and Zr-oxo clusters.

| Molecule | Metal clusters | Position | Energy (kJ · mol⁻¹) |
|---|---|---|---|
| Formic acid | Zr-oxo clusters | A | −14.87 |
| | | B | −8.33 |
| BTB | Zr-oxo clusters | A | −10.51 |

### Example 2. MD Simulations, and Preparation of Sols

To justify the judicious choices of DMF and H₂O as the solvents during synthesis, we firstly conducted MD simulations toward various solvents-nanosheet interactions, as well as interlayer interactions between NUS-8 or NUS-8-NH₂ nanosheets.

### MD Simulations

To provide microscopic insights into the MOF nanosheet-solvents interactions and interlayer interactions between MOF nanosheets, MD simulations were conducted to estimate the binding energies between variable solvents and NUS-8/NUS-8-NH₂ nanosheets, as well as the binding energies between NUS-8/NUS-8-NH₂ interlayers. All the components were described by the Lennard-Jones (LJ) and electrostatic potentials. The LJ potential parameters were adopted from the universal force field (A. K. Rappe et al., J. Am. Chem. Soc. 1992, 114, 10024), and the atomic charges of solvent molecules and relevant nanosheets were estimated using the Qeq method (A. K. Rappe & W. A. Goddard, J. Phys. Chem. 1991, 95, 3358). MD simulations were performed at 298 K for 15 ns. Both NUS-8 and NUS-8-NH₂ structures were assumed to be rigid during MD simulation, whereas all the solvent molecules within nanosheets were considered flexible. The interaction in each model was obtained for the optimized structures after 15 ns of MD simulation. All the LJ interactions were evaluated with a cutoff of 12 Å, and the electrostatic interactions were estimated using the Ewald summation method with an accuracy of 4.18×10.3 kJ·mol⁻¹. Meanwhile, we also calculated the interactions between the formic acid/BTB linker and the metal dusters in different positions using an optimization method in the Forcite module. Additionally, to justify the selective detection of NUS-8-NH₂ sensors, the binding energies of acetone, CO₂, methane, and propane with NUS-8/NUS-8-NH₂/NUS-8-CH₃ nanosheets were also calculated by the same simulation methods and force field parameters.

Preparation of Sols with NUS-8 with Variable Functionalities

Facile solvothermal (120° C.) incubation of DMF and H₂O solutions containing ZrCl₄, H₃BTB, and formic acid (as capping molecule) resulted in the formation of opalescent viscous suspensions/sols.

### Results and Discussion

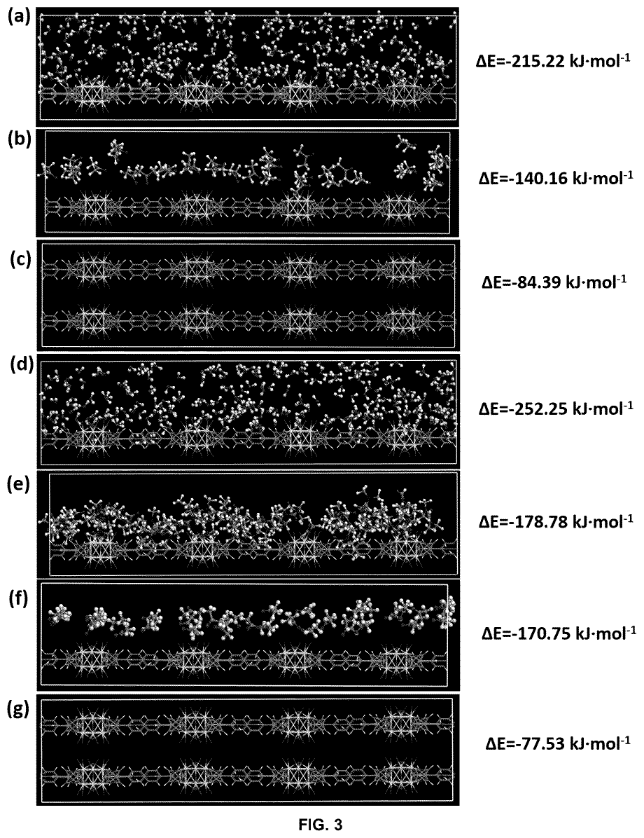

As exemplified in FIGS. 1 and 3, the interactions between DMF/H₂O/acetone and NUS-8 nanosheets far surpass that of NUS-8 interlayer interactions, with respective adsorption energies of −158.24, −215.22, and −140.16 kJ·mol⁻¹ in comparison with −84.39 kJ·mol⁻¹ for NUS-8 interlayer interaction under geometrically optimized configurations. Stronger interactions between the solvent and NUS-8 nanosheets will undoubtedly limit the stacking of the formed nanosheets and thus well stabilize them (M. Zhao et al., Chem. Soc. Rev. 2018, 47, 6267).

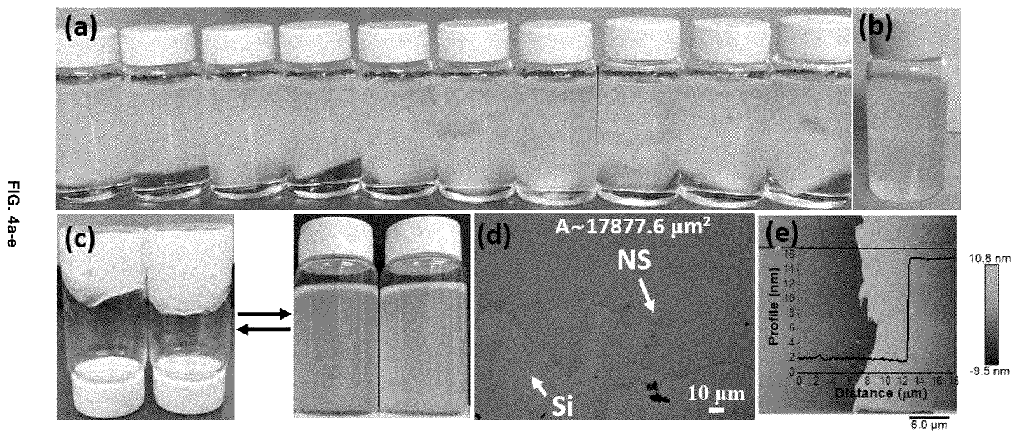

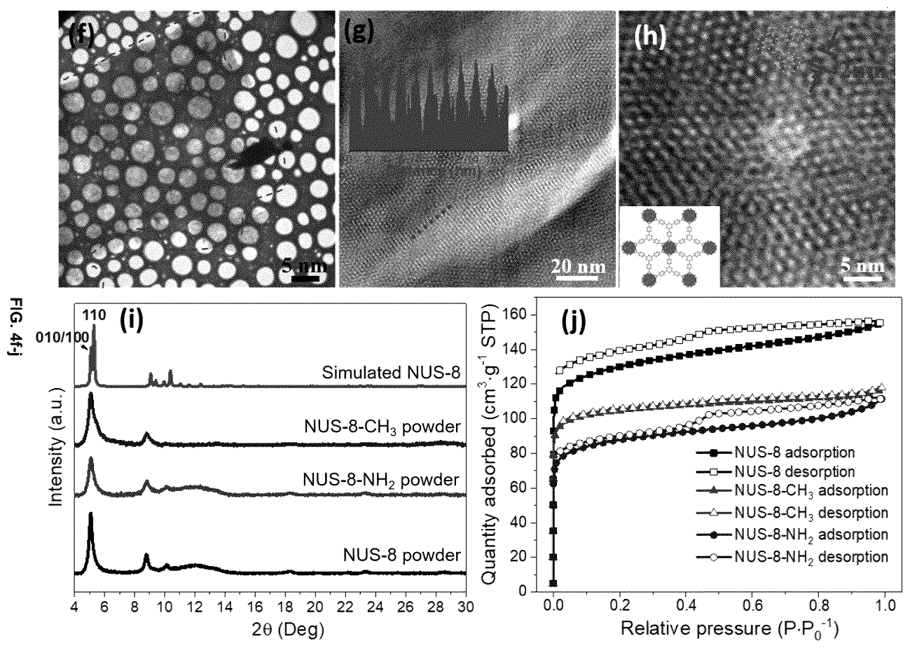

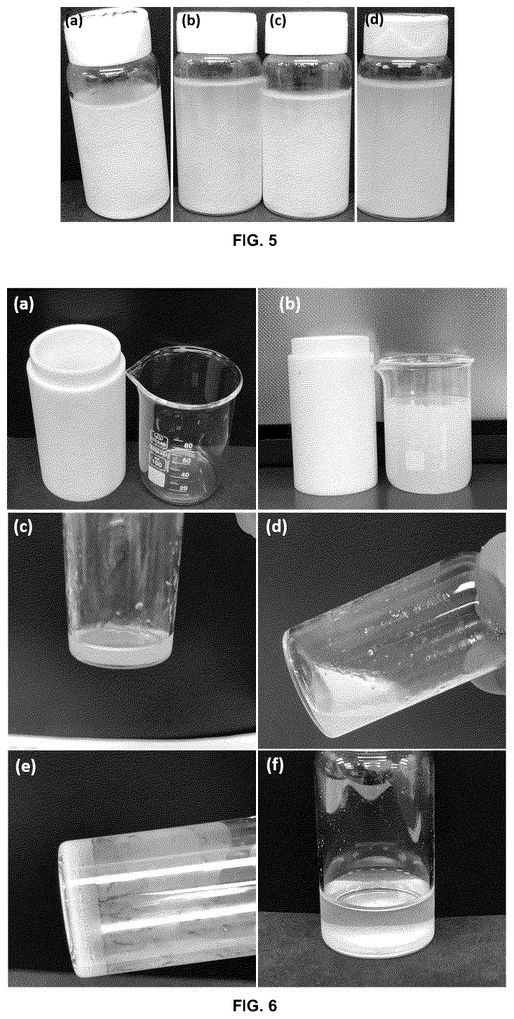

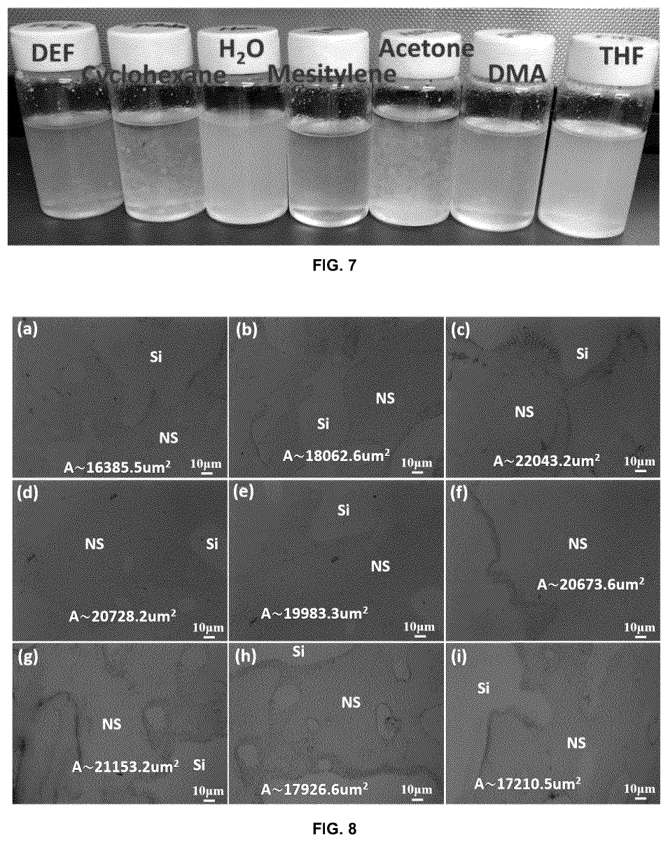

Facile solvothermal incubation of DMF and H₂O solutions containing ZrCl₄, H₃BTB, and formic acid (as capping molecule) resulted in the formation of opalescent viscous suspensions that show Tyndall scattering (FIG. 4a-b). Substitutions of H₂O in precursor solutions by other equivalent solvents like acetone or absolute ethanol also witnessed the formation of viscous suspensions (FIG. 5). However, obvious agglomerations could be noticed in these cases, probably due to the relatively weaker interactions between the solvent and nanosheets (FIG. 3). “Non-flowing” gels can be observed upon removal of the excess solvent via centrifugation. The gels can remain redispersible within varied periods depending on the storage/treatment conditions (FIG. 6c-f). Rapid phase transformation from non-dried gel to sol happened once fresh solvent like DMF, H₂O, or THF was added, and vice versa (FIG. 4c and 7). Moreover, the resultant sols were stable over two years without precipitation.

Thus, the direct, scalable, and surfactant-free synthesis of stable MOF suspensions encompassing exceptionally large NUS-8 nanosheets with variable functionalities and excellent solution processability, simply via adding capping molecules, as well as judicious controls of precursor concentration and MOF nanosheets-solvent interactions was demonstrated.

### Example 3. Characterization of NUS-8, NUS-8-CH₃, and NUS-8-NH₂Nanosheets and Sols

Estimated Areas of NUS-8 Nanosheets with Variable Functionalities

A rather diluted sol (<10 g·L⁻¹, NUS-8, NUS-8-CH₃ or NUS-8-NH₂) was drop-casted onto atomically-flat Si surfaces. The areas of NUS-8, NUS-8-CH₃, and NUS-8-NH₂ nanosheets deposited onto Si surfaces were estimated using the processing software Image J.

### X-Ray Absorption Fine Structure (XAFS)

XAFS spectra were recorded in transmission mode at room temperature at the XAFCA beamline of the Singapore Synchrotron Light Source.

### ATR-FTIR

ATR-FTIR patterns were obtained by a Bio-Rad FTS-3500 ARX FTIR spectrometer. The NUS-8 nanosheets were dispersed in ethanol, and “non-flowing” NUS-8 gel was obtained by removing excess ethanol by centrifugation. The gel was intermittently heated at 60° C. for 5 min and afterward measured by ATR-FTIR.

### Preparation of NUS-8(Hf) Powder and Sol

The synthesis of NUS-8(Hf) suspensions/sols was conducted at 120° C. for 24 h in mixture solutions containing HfCl₄ (30 mg) and H3BTB-NH₂ (30 mg). The solution was composed of dimethylformamide (DMF, 15 mL), ultrapure water (2 mL), and formic acid (2 mL). NUS-8(Hf) powder was fabricated by drying 15 mL of NUS-8(Hf) alcoholic suspension with a concentration of 4 g·L⁻¹ at 25° C. under vacuum.

### Results and Discussion

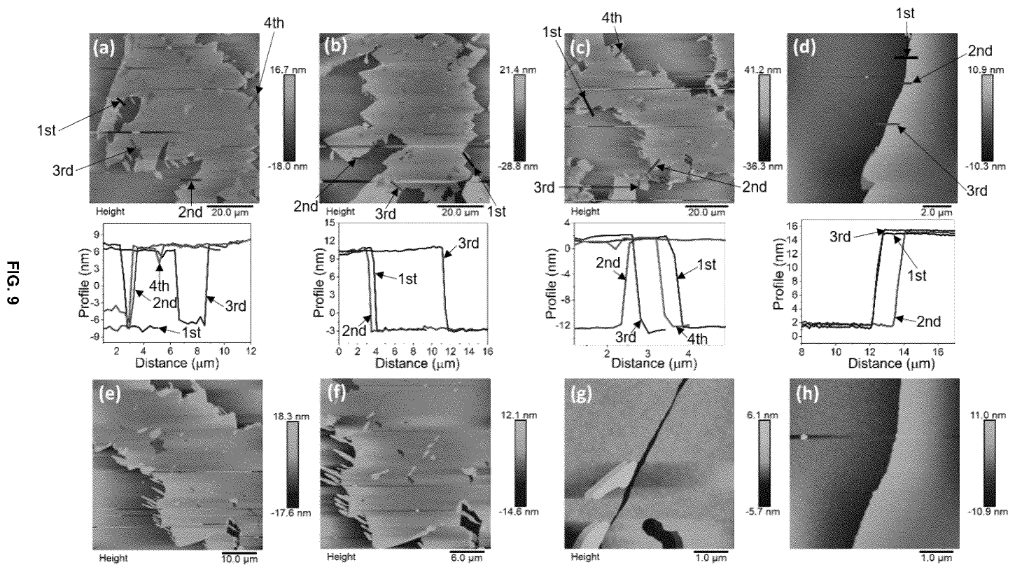

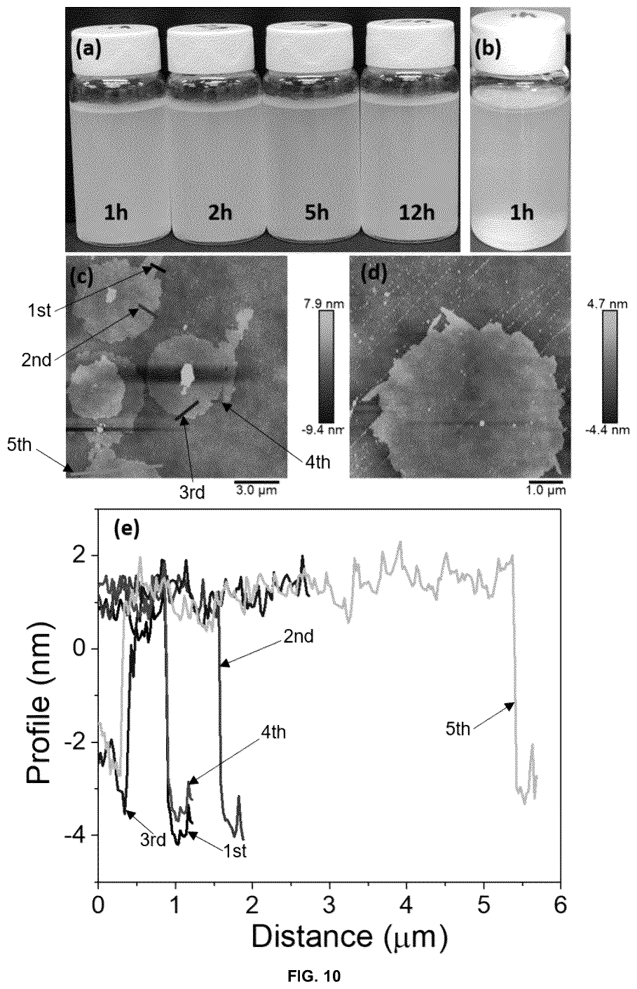

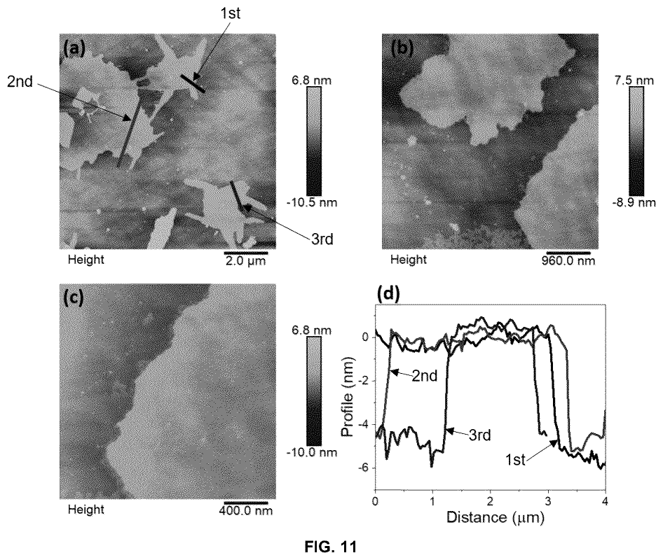

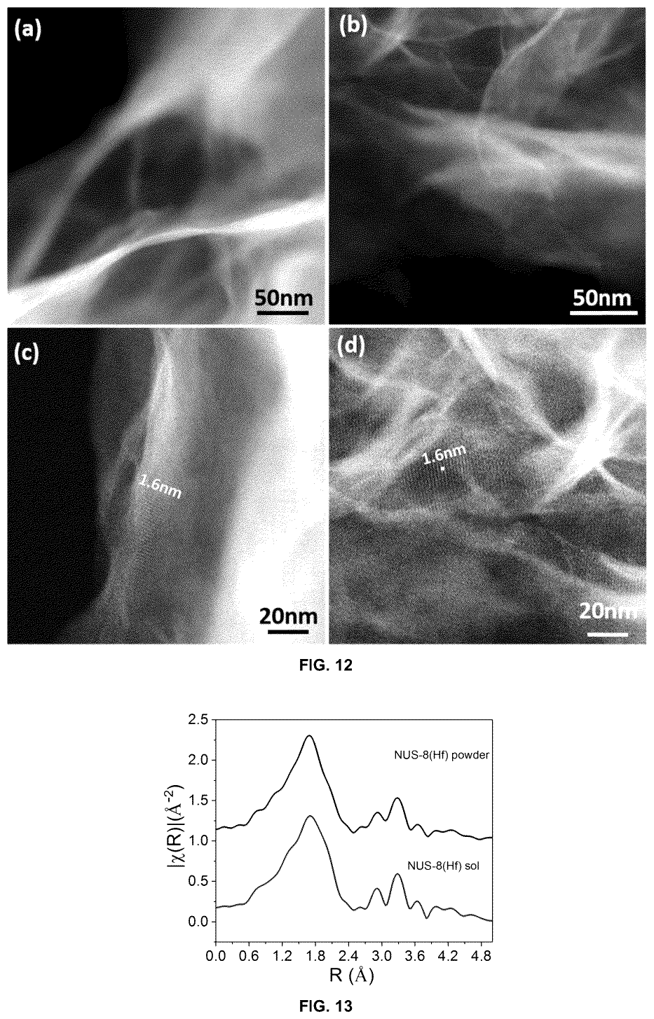

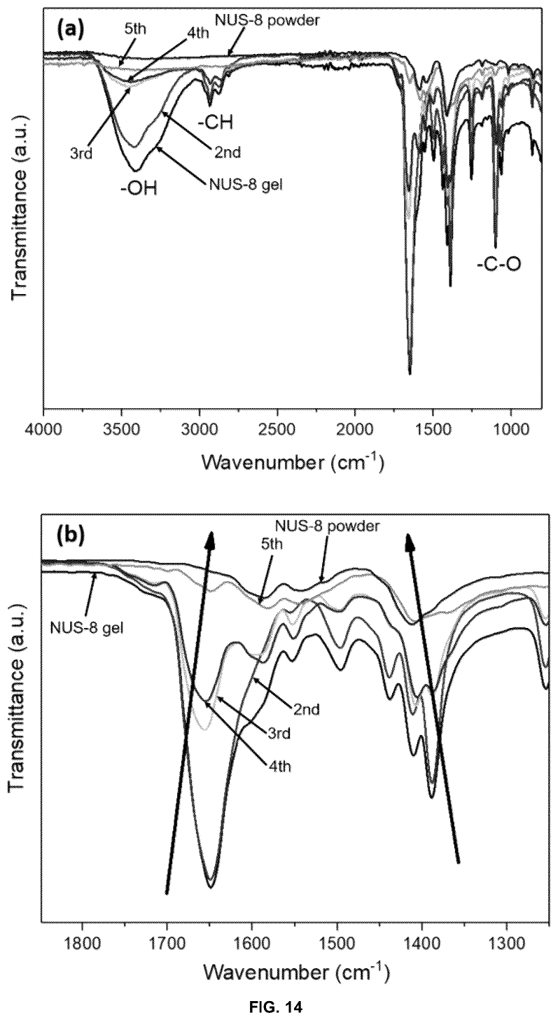

SEM imaging (FIG. 4d) confirms the formation of large-area (>17877.6 μm², measured by Software Image J) and monodispersed nanosheets obtained via drop-casting of a rather diluted sol onto atomically-flat Si surfaces. Additional checking of 9 more areas gave similar results (FIG. 8). The average area of the ten fields of view captured is 19204.4±1832.0 μm². This is distinct from literature reporting the formation of metal-organic nanosheets (MONs) with areas commonly less than hundreds of μm². Although the interface-mediated and Langmuir-Blodgett approaches can also allow the formation of large-area MONs, our approach presented here outstands in scale-up production (M. Zhao et al., Chem. Soc. Rev. 2018, 47, 6267; and Y.-Z. U, Z.-H. Fu & G. Xu, Coord. Chem. Rev. 2019, 388, 79). AFM (FIG. 4e and 9) and low-magnification TEM images (FIG. 4f) manifest the 2D crystallographic nature of NUS-8 nanosheets, whose thicknesses are all around 13 nm from their height profiles obtained by AFM. HAADF-STEM images of the nanosheet further affirm its lattice fringes with one representative interplanar spacing of 1.6 nm (FIG. 4g-h), corresponding to the (010)/(100) crystallographic facet of NUS-8 (R. Wang et al., Inorg. Chem. 2014, 53, 7086; Z. Hu et al., J. Mater. Chem. A 2017, 5, 8954; and Z.-R. Tao et al., Nat. Commun. 2019, 10, 2911). Interestingly, unambiguous kagome dual nets with bright spots representing the Zre clusters can be observed (FIG. 4h). The ordering of Zre clusters with two adjacent ones separated by a distance of 2 nm matches well with the theoretical atomic model as indicated in the inset of FIG. 4h. Simultaneously, the XRD pattern of the obtained powder is consistent with that of the calculated one, suggesting the formation of NUS-8 phase (R. Wang et al., Inorg. Chem. 2014, 53, 7086; and Z. Hu et al., J. Mater. Chem. A 2017, 5, 8954). Time-dependent investigations on its formation mechanism clearly showed that thinner NUS-8 MONs can form under shorter growth periods (FIG. 10-12). The generated NUS-8 nanosheets gradually formed a sol state through a weakly, non-covalently bonded colloidal network structure throughout the liquid volume, which adopts the shape of the container (J. Hou, A. F. Sapnik & T. D. Bennett, Chem. Sci. 2020, 11, 310). The non-covalent interactions between discrete nanosheets, as well as between nanosheets and the solvent are mainly van der Waals forces, reflected by EXAFS characterizations toward the NUS-8 powder (dry form) and NUS-8 sol (wet form), and ex-situ ATR-FTIR measurements conducted on the “non-flowing” NUS-8 gels under intermittent heating (FIG. 13-14). The ATR-FTIR results confirm the existence of the solvent within the “non-flowing” NUS-8 gel. Therefore, interactions between NUS-8 nanosheets and the solvent, and between discrete nanosheets can be feasibly anticipated. The resulting opalescent suspensions displayed similar property as shown previously. Such observation justifies the strategy mentioned above, and the successful synthesis of NUS-8 suspension in large volume (FIG. 6a-b) further highlights the advantages of this bottom-up approach in the scalable synthesis of ultrathin MONs.

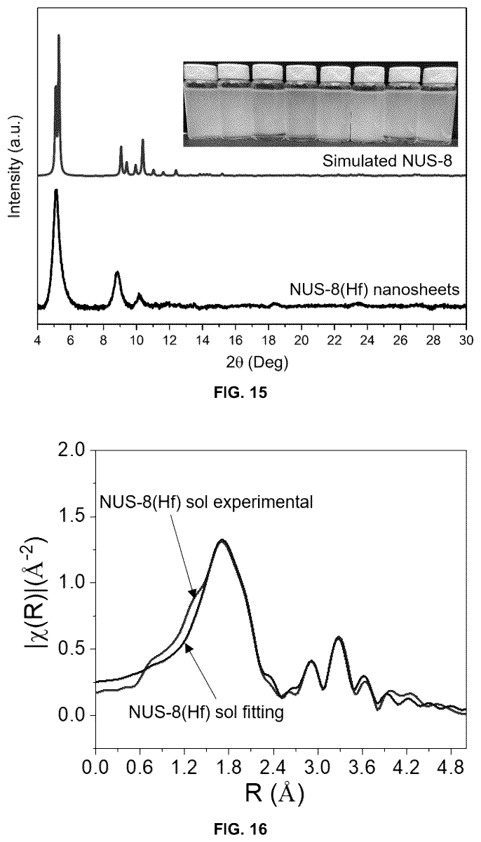

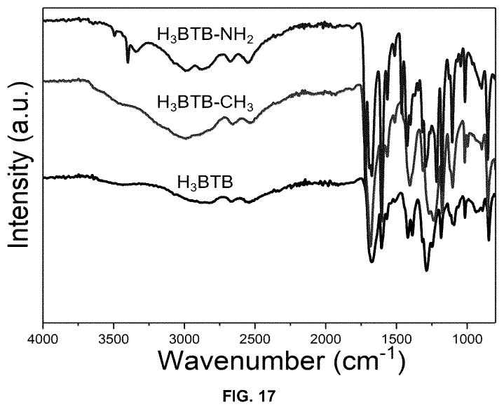

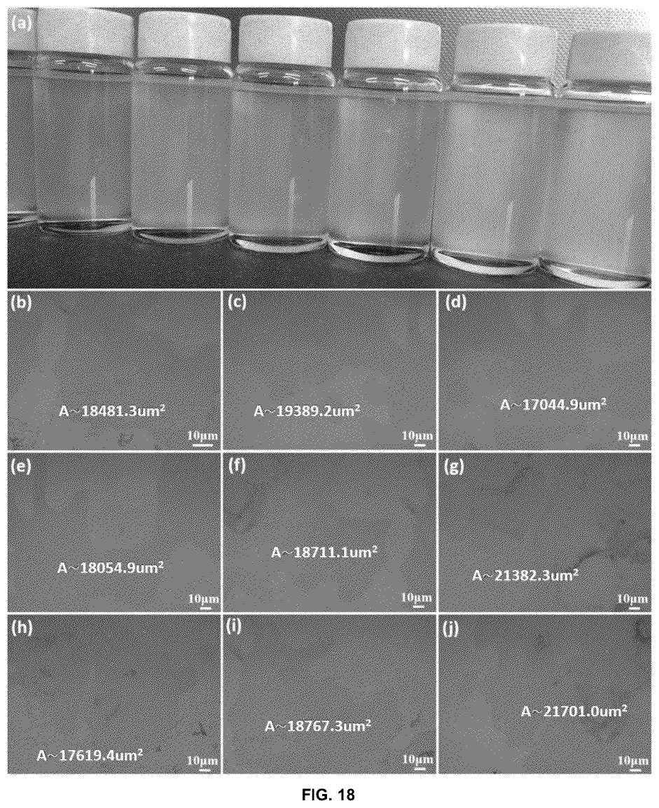

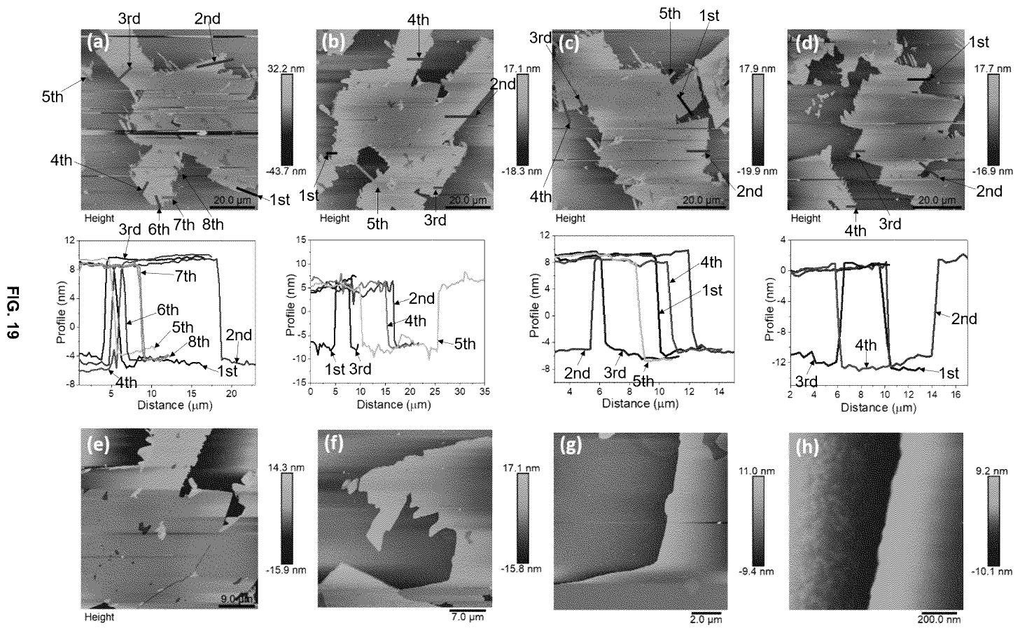

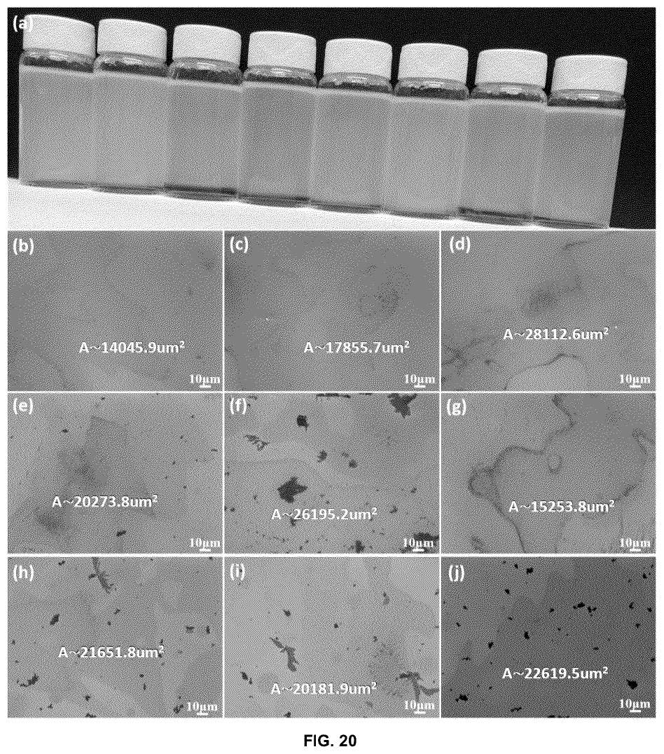

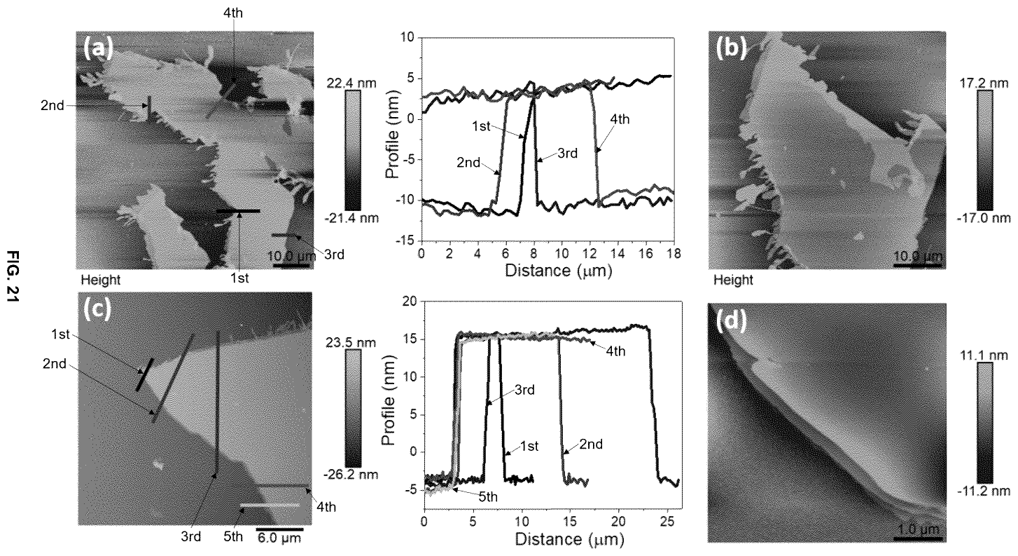

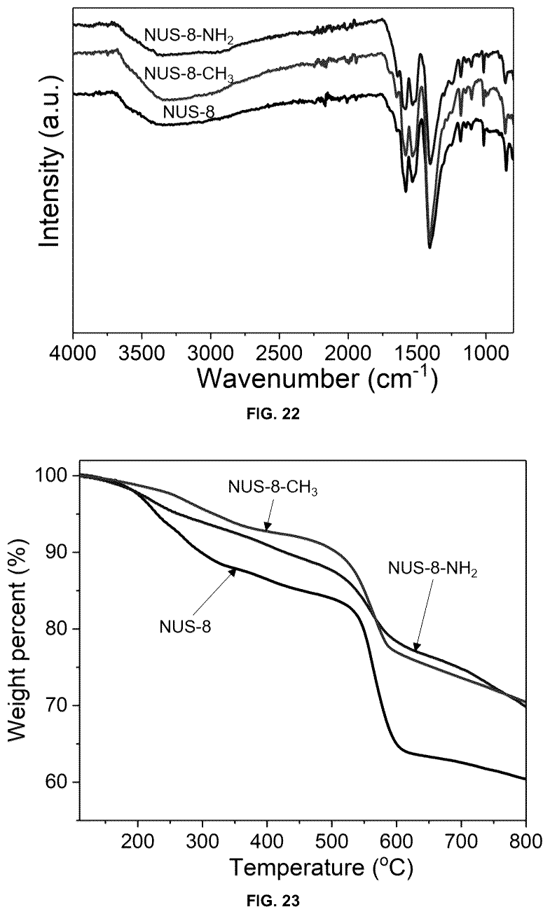

Alternatively, the same phase can also be obtained using Hf salt as the metal source (FIG. 15). The SBUs of the synthesized NUS-8(Hf) were further verified by X-ray absorption spectroscopy (FIG. 16). Fitting of the EXAFS profile of the NUS-8(Hf) nanosheets implies that Hf⁴⁺ ions are coordinated with eight oxygen atoms in the SBUs, with Hf-O (carboxylate) and Hf-(μ₃-O) distances of 2.11±0.01 and 2.27±0.02 Å, respectively. ¹H NMR spectrum of digested NUS-8 nanosheets in D₂O also corroborates the existence of formate, likely coordinated with Hf. The molar ratio of formate to BTB is deduced to be 1, justifying the above hypothesis that the coordination of capping molecules with SBUs out of 2D plane endows the formation of large-area MONs. Likewise, viscous suspensions composed of large-area NUS-8 nanosheets with variable terminated functional groups like—CH₃ and —NH₂ in comparable thickness and area have also been successfully synthesized based on the strategies described in Example 1 (FIG. 4i and 17-23). The Brunauer-Emmett-Teller (BET) surface areas were computed to be 404.99, 321.62, and 274.12 m² g⁻¹ for NUS-8, NUS-8-CH₃, and NUS-8-NH₂, respectively, on account of N₂ sorption measurements conducted at 77 K (FIG. 4j). Those values are lower than the reported ones of their 3D counterparts (613 m² g⁻¹, R. Wang et al., Inorg. Chem. 2014, 53, 7086) and 2D Zr-BTB MONs (570 m² g⁻¹) with smaller nanosheet sizes (Z. Hu et al., J. Mater. Chem. A 2017, 5, 8954).

### Example 4. NUS-8 Monoliths, Aerogels and Xerogels

Shaping MOFs into macro- or mesoscopically structured objects with hierarchical pore architectures is highly desirable to expand their applicability in industrial scenarios. The direct usage of micro/nanometered MOF powders is confronted with several technical challenges such as poor handling, limited mass transfer, dust formation, and unwanted pressure drops in packed beds (B. Bueken et al., Chem. Sci. 2017, 8, 3939; and J. Hou, A. F. Sapnik & T. D. Bennett, Chem. Sci. 2020, 11, 310). In this regard, the synthesized suspensions composed of large-area NUS-8 MONs with tunable functionalities exhibit excellent solution processability. Thus, they can be facilely processed into monoliths, xerogels, or aerogels without binders, and even homogeneous and textured films with adjustable thickness.

### Fabrication of NUS-8 Monoliths

Ethanolic NUS-8 suspension (prepared in Example 1, 30 mL, 8 g·L⁻¹) was pre-dried at 80° C. for 10 min to remove most of the solvent, and then the non-flowing gel was left in the fumehood (˜20° C., 60% relative humidity) for slow drying. NUS-8 monoliths with variable shapes can be feasibly obtained resting with the shape of the used container.

### Fabrication of NUS-8 Aerogels

Aqueous NUS-8 suspension (prepared in Example 1, 20 mL, 4 g·L⁻¹) was firstly frozen at −70° C.˜−80° C. by freezing-drier. Then, the solution was primarily dried under reduced pressure (0.013 mbar) by sublimation. Finally, in the secondary drying process, the residual solvent was fully extracted using ALPHA2-4 LD plus, resulting in the formation of NUS-8 aerogels.

### Fabrication of NUS-8 Xerogels

NUS-8 xerogel was fabricated by drying ethanolic NUS-8 suspension (prepared in Example 1, 15 mL, 4 g·L⁻¹) at 25° C. under vacuum. The procedure used in the fabrication of NUS-8 monolith can also induce the formation of xerogels.

### CO₂ Adsorption Isotherms

CO₂ isotherms of NUS-8 aerogels and xerogels were collected by a Quantachrome Autosorb iQ.

### Characterization

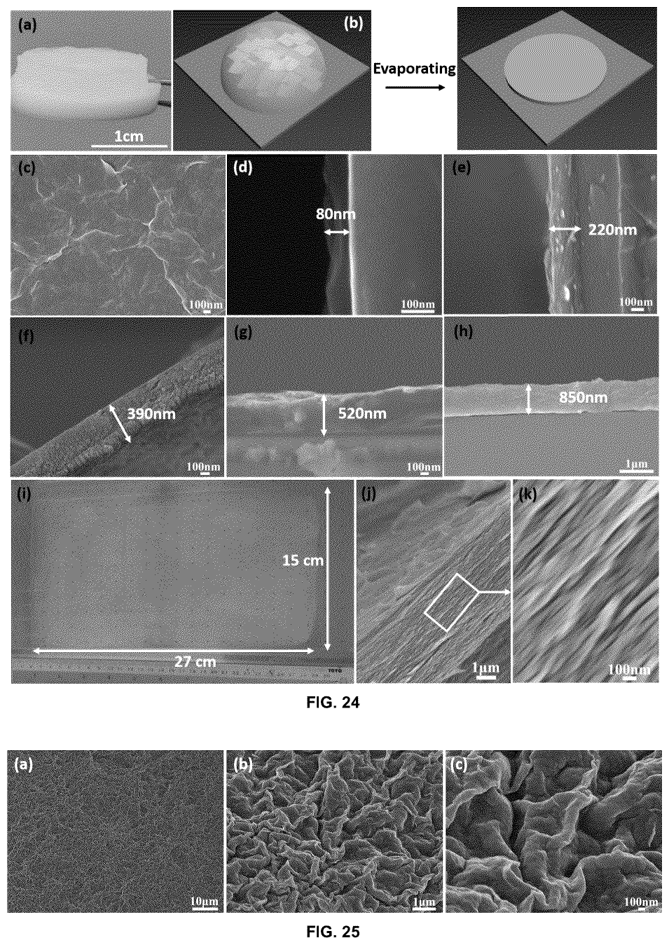

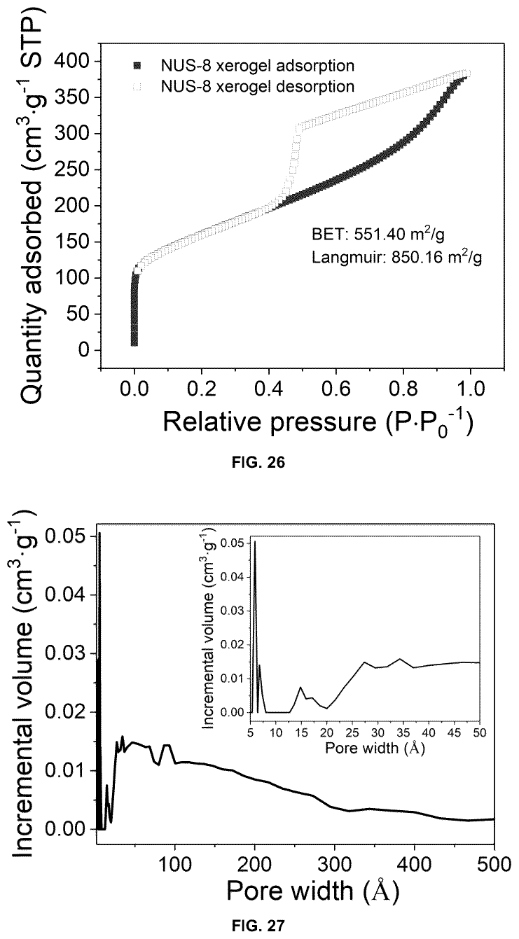

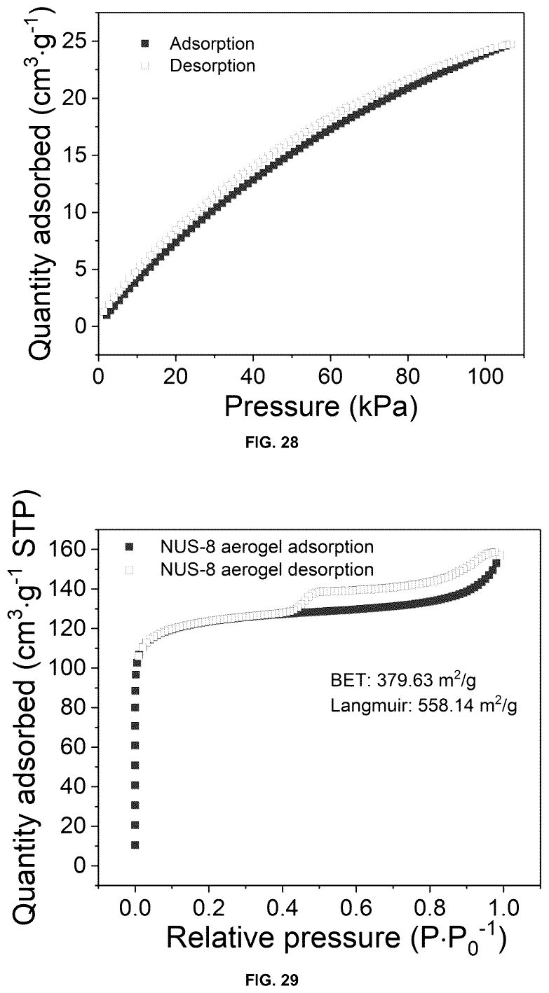

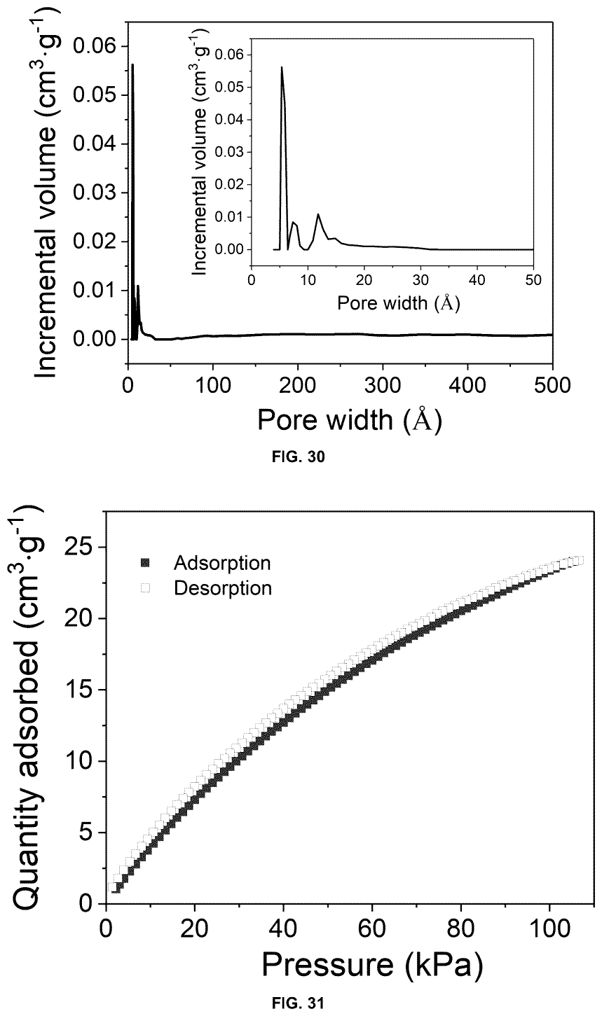

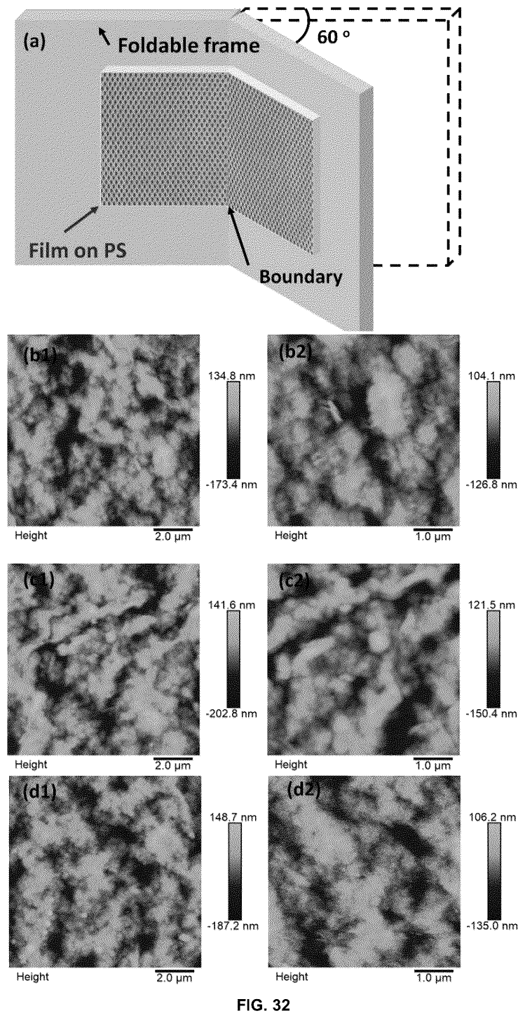

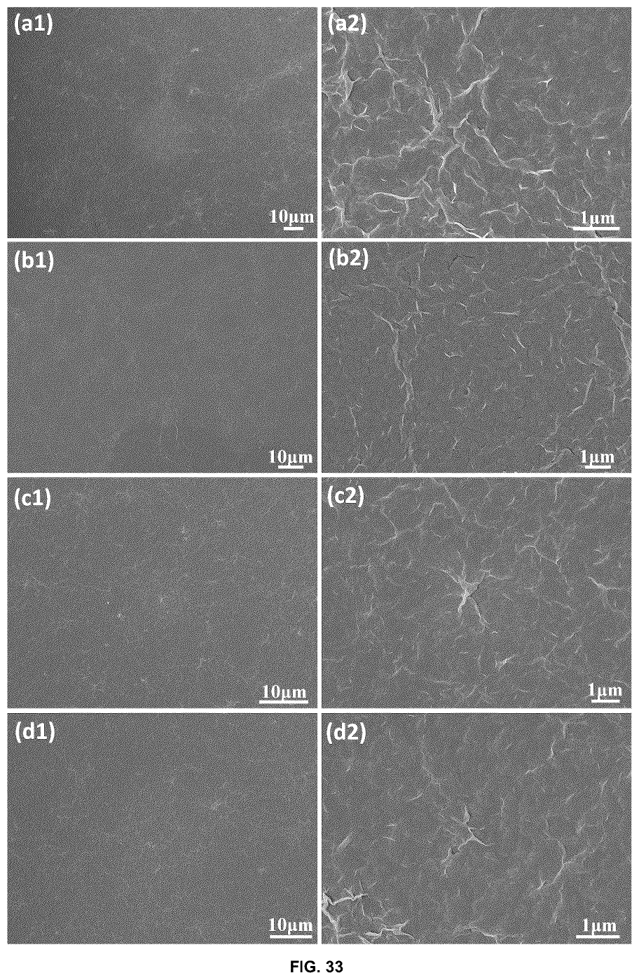

FIG. 24a shows the optical photograph of NUS-8 monolith fabricated from the non-flowing gel upon slow solvent removal under ambient conditions. The primary gel experienced significant shrinkage in volume because of the capillary forces exerted dung ethanol evaporation. Winkled sheet-like architectures can be observed (FIG. 25). Alternatively, NUS-8 xerogels or aerogels with modulable sorption behaviors can also be achieved simply by controlling the drying process (FIG. 26-31). Obviously, differentiated mesoporosity can be feasibly introduced within NUS-8 xerogels/aerogels while preserving the original microporosity. This fabrication method outperforms the conventional approaches concerning the macroscale and architectural shaping of MOF objects that commonly rely on either structural templating of an exotic phase or pelletizing MOF powder via extrusion or mechanical compression under a fine-tuned pressure (G. W. Peterson et al., Microporous Mesoporous Mater. 2013, 179, 48; and B. Van de Voorde et al., J. Mater. Chem. A 2015, 3, 1737).

As proof-of-principle demonstrations, NUS-8 monoliths, aero- and xerogels, and large-area homogeneous films with texture, controllable thickness, and appreciable mechanical properties can be facilely fabricated by its good solution processability and 2D nature irrespective of the substrate. This approach highlights the importance of solution processability in the synthesis of extended porous functional solids, and opens up novel perspectives for their easy integrations and mass-production of MOFs in miniaturized electronic devices with realistic mass production targeted for chemical sensing and low-k dielectrics/optoelectronics, and membrane-based separation technologies.

### Example 5. NUS-8 Films on Si and PS Substrates, and Si Wafer

A crucial step to fulfill the full potentials of MOFs in electronics, optoelectronics, and membrane-based separations lies in the easy fabrication of MOF films onto relevant surfaces with excellent homogeneity, compactness, and preferential orientation (O. Shekhah et al., Chem. Soc. Rev. 2011, 40, 1081; and I. Stassen et al., Chem. Soc. Rev. 2017, 46, 3185). NUS-8 films on small substrates were fabricated just via drop-casting.

### Fabrication of NUS-8 Films on Si Substrates

Typically, Si substrates were thoroughly rinsed by absolute ethanol twice under fierce stirring with each cycle for 10 min, then dried at 80° C. under ambient pressure. A certain volume of NUS-8 (prepared in Example 1) aqueous suspension (C_{NUS-8}=0.17 g·L⁻¹, 10 μL, C_{NUS-8}=0.5 g·L⁻¹, 10 μL, C_{NUS-8}=1 g·L⁻¹, 10 μL, C_{NUS-8}=1 g·L⁻¹, 20 μL, or C_{NUS-8}=1 g·L⁻¹, 30 μL) was then captured by pipette and vertically dropped onto the Si surfaces. NUS-8 films can thus be formed along with the vaporization of water.

### Fabrication of NUS-8 Film on PS Substrate

PS substrate was rinsed by air plasma in a Harrick plasma cleaner for 4 min. The pre-treated PS substrate was placed on an automatic film coater. Viscous NUS-8 (prepared in Example 1) ethanolic suspension (8 mL, 3.2 g·L⁻¹) was dropped by pipette onto the PS substrate. Then, doctor-blading with a set height of 50 μm was performed at a speed of 1.3 cm·s⁻¹ on the PS substrate. Drying of the film was conducted at an ambient condition to fully vaporize the solvent, yielding a flat film. Further activations were done at 120° C. under vacuum.

### Wafer Fabrication of NUS-8 Film

The 8-inch wafer fabrication of NUS-8 film was prepared from a NUS-8 (prepared in Example 1) ethanolic suspension (8 mL, 3.3 g·L⁻¹) and a 8-inch Si wafer by following the protocol for fabrication of NUS-8 film on PS substrate except under a set height of 30 μm.

### Folding Tests

The film was placed on a foldable frame as depicted in FIG. 32. The folding process was repeated for 100 and 200 times, respectively. The foldable angle was kept at 60° during each circle.

### Results and Discussion

Because of the excellent solution processability of the resultant suspensions and the large area of NUS-8 nanosheets, a series of compact and homogeneous NUS-8 films can be easily fabricated onto Si surfaces via simple drop-casting (FIG. 24b-h and 33). Films with variable thicknesses ranging from 80 to 850 nm can be achieved by varying the concentration of the suspension, drop volume, and hydrophobicity/hydrophilicity of substrates. The fluidic feature of the resultant stable NUS-8 suspensions with variable functionalities can also be harnessed to produce large-area (27×15 cm² and 8-inch wafer) and textured MOF films. Films fabricated onto various substrates like PS and Si wafer using shear force as in doctor-blading showed great homogeneity (FIG. 24i-k and 34a). Folding tests toward the NUS-8 film fabricated on the flexible PS substrate affirmed its robustness and processability even after 200 times' folding (FIG. 32), holding tremendous potentials in practical applications.

### Example 6. Crystallinity, Orientation, and Physical Properties of NUS-8 Films

### Mechanical Tests

Nanoindentation tests upon the NUS-8 film on Si were conducted by a nanoindenter on XE 70-AFM equipped with a cone diamond tip with a spring constant of 42 N·m⁻¹ under contact mode. The radius of the tip was around 10 nm, and the scanning speed was 1 Hz. Use of the XEI Image Processing software gives estimates of Young's modulus from force-displacement curves through several steps. The first step concerns removing any offset or tilt from the curve and finds the contact point. The second step subtracts the cantilever bending from the piezo movement to yield the indentation. Finally, the Hertz model can be applied, and the geometry of the indenter, as well as Poisson's ratio, is to be specified, both of which are available on hand. Note that the measurements were conducted at multiple points. A grid of 16 squares was measured, and the adhesion forces were determined for the center of each square.

### Scotch Tape Tests

The thickness of the film was 400 nm. A piece of tape was adhered to the film and then peeled off; this process was repeated thrice.

### Results and Discussion

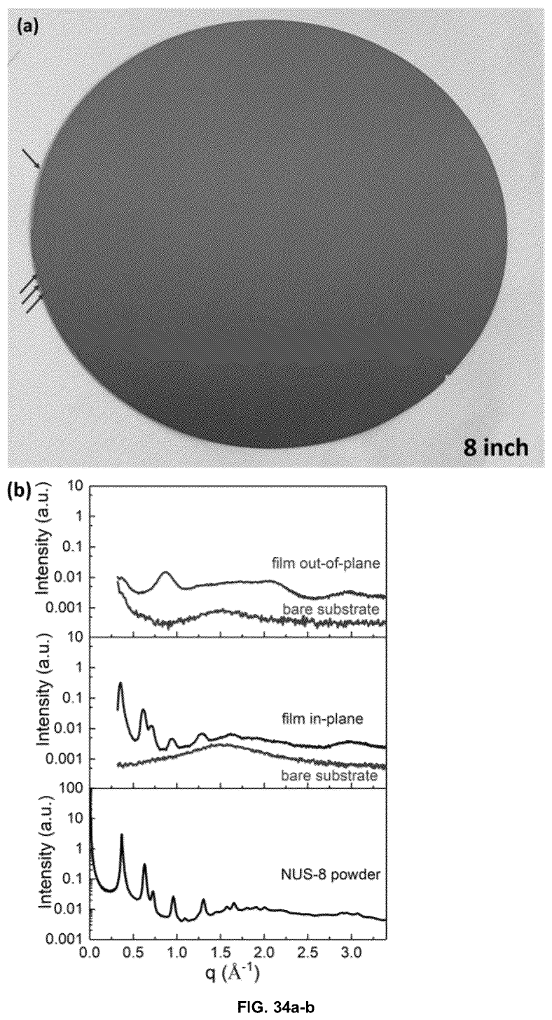

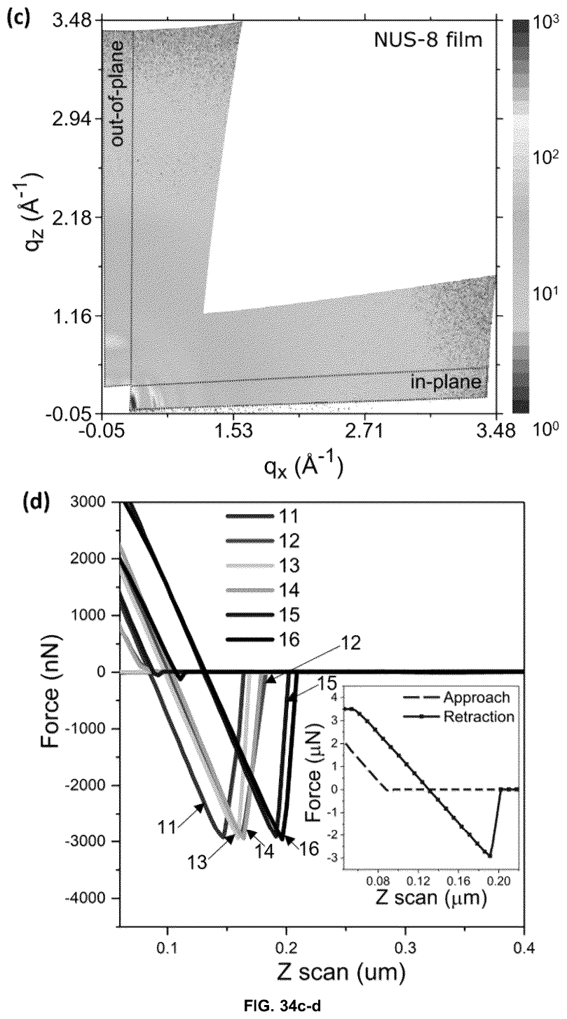

2D-GIWAXS measurements were conducted to further check the crystallinity and orientation of relevant films (FIG. 34b-c). Noticeably, the obtained NUS-8 film only gave a broad out-of-plane peak, manifesting its preferential orientation with the [001] direction perpendicular to the substrate. This complies with the arrangement of NUS-8 nanosheets that are parallel to the surface (Z.-R. Tao et al., Nat. Commun. 2019, 10, 2911). The interlayer spacing was 0.7 nm, and the apparent broadening of the peak is attributed to the weak stacking interactions between adjacent layers. Comparatively, in-plane measurement toward the film showed nearly all the peaks identical to that of the bulk NUS-8 powder, indicating no in-plane orientation. The observed out-of-plane orientation of NUS-8 nanosheets endows their fragmented one-dimensional pore channels perpendicular to the substrate surface and is therefore beneficial to gas uptake (O. Yassine et al., Angew. Chem. Int. Ed. 2016, 55, 15879; and M.-S. Yao et al., Adv. Mater. 2016, 28, 5229). Moreover, understanding the fundamental MOF mechanical structure-property relationship is a prerequisite for device-based technological applications.

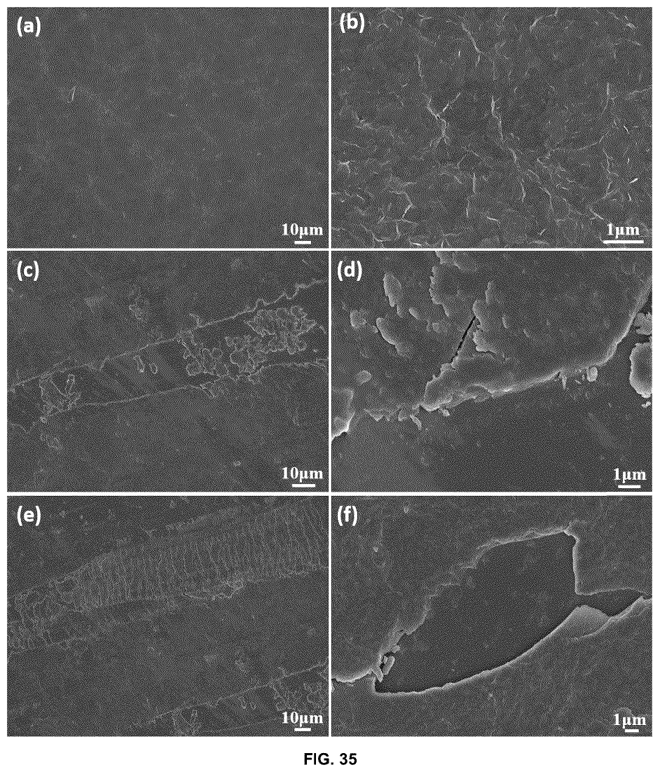

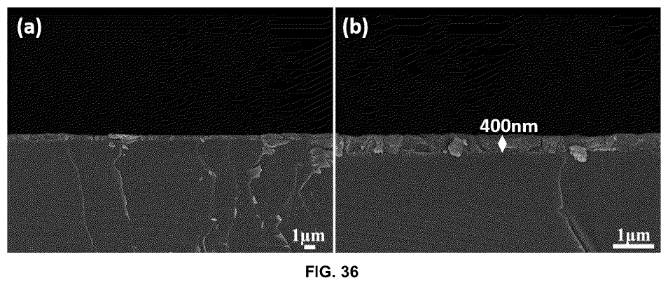

To gain more insights into the mechanical properties, including Young's modulus and adhesion energy, AFM-based nanoindentation tests were performed. FIG. 34d depicts the representative force-displacement curves collected between a diamond AFM tip and the NUS-8 film. Fitting the force-displacement curves with Hertzian models of contact using the XEI image processing software gives estimates of Young's modulus and adhesion energy of the film (F. Variola, Phys. Chem. Chem. Phys. 2015, 17, 2950; and F. M. Rusu et al., IOP Conf. Ser.: Mater. Sci. Eng. 2016, 147, 012023). The averaged Young's modulus and adhesion energy of 16 points collected within the (001) plane of the NUS-8 film were 1.6±0.05 GPa and 4.32±1.06 J·m⁻², respectively. The resultant value of Young's modulus is comparable with those of other MOFs from the literature (Table 2, N. C. Burtch et al., Adv. Mater. 2018, 30, 1704124). The slightly larger adhesion energy value than that of ZIF-8 film (Z. Zeng & J.-C. Tan, ACS Appl. Mater. Interfaces 2017, 9, 39839) and additional scotch tape tests (FIG. 35) on the film clearly indicate that the film well adheres to the underneath substrate. It was found that multiple scotch tape tests could indeed remove partial NUS-8 film, whereas part of the film remains intact. More impressively, clear-cut ‘footprints’ of the removed NUS-8 film can be noticed after the scotch tape test (FIG. 35c-f), suggesting a decent adhesion between the converted NUS-8 film and the substrate.

TABLE 2
Comparison of Young's modulus of different MOFs. Note that
Young's modulus of MOFs is highly dependent on their chemical
structure, network topology, porosity, crystallographic
facet, external molecules captured, and temperature.

| | Young's modulus | | |
|---|---|---|---|
| MOF type | (GPa) | Method | Reference |
| MOF-5 | 2.7 ± 1.0 | Nanoindentation on | D. F. Bahr et al., |
| | | single crystal | Phys. Rev. B 2007, |
| | | | 76, 184106 |
| ZIF-4 | 4.6 ± 0.4 | Nanoindentation on | J. C. Tan, T. D. |
| | | single crystal | Bennett & A. K. |
| | | | Cheetham, Proc. |
| | | | Natl. Acad. Sci. |
| | | | U.S.A. 2010, 107, |
| | | | 9938 |
| ZIF-7 | 6.5 ± 0.5 | Nanoindentation on | J. C. Tan, T. D. |
| | | single crystal | Bennett & A. K. |
| | | | Cheetham, Proc. |
| | | | Natl. Acad. Sci. |
| | | | U.S.A. 2010, 107, |
| | | | 9938 |
| ZIF-8 | 3.2 ± 0.2 | Nanoindentation on | J. C. Tan, T. D. |
| | | single crystal | Bennett & A. K. |
| | | | Cheetham, Proc. |
| | | | Natl. Acad. Sci. |
| | | | U.S.A. 2010, 107, |
| | | | 9938 |
| ZIF-9 | 6.0 ± 0.4 | Nanoindentation on | J. C. Tan, T. D. |
| | | single crystal | Bennett & A. K. |
| | | | Cheetham, Proc. |
| | | | Natl. Acad. Sci. |
| | | | U.S.A. 2010, 107, |
| | | | 9938 |
| ZIF-20 | 3.8 ± 0.4 | Nanoindentation on | J. C. Tan, T. D. |
| | | single crystal | Bennett & A. K. |
| | | | Cheetham, Proc. |
| | | | Natl. Acad. Sci. |
| | | | U.S.A. 2010, 107, |
| | | | 9938 |
| ZIF-68 | 3.5 ± 0.4 | Nanoindentation on | J. C. Tan, T. D. |
| | | single crystal | Bennett & A. K. |
| | | | Cheetham, Proc. |
| | | | Natl. Acad. Sci. |
| | | | U.S.A. 2010, 107, |
| | | | 9938 |
| ZIF-zni | 9.0 ± 0.3 | Nanoindentation on | J. C. Tan, T. D. |
| | | single crystal | Bennett & A. K. |
| | | | Cheetham, Proc. |
| | | | Natl. Acad. Sci. |
| | | | U.S.A. 2010, 107, |
| | | | 9938 |
| CuBDC | 23.0 | AFM-based | Z. Zeng, I. S. |
| | | nanoindentation on | Flyagina & J.-C. |
| | | CuBDC nanosheet | Tan, Nanoscale |
| | | | Adv. 2020, 2, 5181 |
| CuBTC | 6.5 ± 0.9 | Nanoindentation on | J. Heinen et al., |
| | | single crystal | ACS Appl. Mater. |
| | | | Interfaces 2018, 10, |
| | | | 21079 |
| NUS-8 | 1.6 ± 0.05 | AFM-based | This work |
| | | nanoindentation on | |
| | | NUS-8 film | |

1. A method of forming a metal-organic framework (MOF) suspension, the process comprising:
(a) providing a metal precursor, an organic ligand, a capping molecule and a solvent comprising a polar aprotic solvent and water;
(b) adding the metal precursor, organic ligand and capping molecule to the solvent to provide a reaction mixture and heating it for a period of time to form the MOF suspension, wherein:
the metal precursor has a concentration in the solvent of from 1.0 to 2 mg/mL;
the organic ligand has a concentration in the solvent of from 1.0 to 2 mg/mL;
the capping molecule is provided in an amount of from 5 to 20 vol % relative to the total volume of the solvent, and/or it is provided in an amount of from 5 to 20 wt % relative to the total weight of the solvent; and
water represents from 5 to 20 vol % of the solvent, with the balance being the polar aprotic organic solvent.

2. The method according to claim 1 further comprising the purification steps of:
(a) removing the MOF from the reaction mixture by centrifugation;
(b) adding a polar aprotic solvent to the removed MOF to form a suspension and then removing the MOF by centrifugation;
(c) repeating step (b) from one to ten times; and
(d) adding water to the removed MOF to form a suspension and then removing the MOF by centrifugation and repeating from two to ten times (e.g. from 4 to 6 times, such as five times) over a 24-hour period;
(e) adding water and/or tetrahydrofuran to the removed MOF to provide a final suspension of the MOF.

3. The method according to claim 1, wherein the polar aprotic solvent is selected from one or more of N,N-dimethylformamide (DMF), acetone, acetonitrile, dimethyl sulfoxide, dimethylacetamide, N,N-diethylformamide.

4. The method according to claim 1, wherein the capping molecule is provided in an amount of from 10 to 15 vol % relative to the total volume of the solvent, and/or it is provided in an amount of from 10 to 15 wt/o relative to the total weight of the solvent.

5. The method according to claim 1, wherein the metal precursor is selected from one or more of ZrOX₂, HfOX₂, ZrX₄, HfX₄, AlX₃, CrX₃, FeX₃, and TiX₄, where X is halo selected from Cl, Br, I, or F.

6. The method according to claim 1, wherein the organic ligand is a tridentate carboxylic acid that does not incorporate further functional groups or is a tridentate carboxylic acid that incorporates further functional groups (e.g. from 1 to 10, such as from 1 to 5, such as from 1 to 3 further functional groups).

7. The method according to claim 1, wherein the capping molecule is a monodentate carboxylic acid.

8. (canceled)

9. A suspension of a metal organic framework comprising:
one or both of water and tetrahydrofuran (THF); and
MOF nanosheets having an area of greater than or equal to 10,000 μm2.

10. A sol comprising:
a solvent; and
MOF nanosheets having an area of greater than or equal to 10,000 μm².

11. A composition comprising MOF nanosheets having an area of greater than or equal to 10,000 μm².

12. (canceled)

13. The suspension according to claim 9, wherein the MOF in the MOF nanosheets is selected from one or more of NUS-8 and/or an analogue thereof, where each MOF is formed from:
(a) a metal ion and an organic ligand; or
(b) a metal ion and an organic ligand and a capping molecule.

14. (canceled)

15. (canceled)

16. (canceled)

17. The suspension according to claim 9, wherein the MOF is NUS-8 or an analogue thereof having:
(a) Zr⁴⁺ as the metal ion, benzene-1,3,5-tribenzoate as the organic ligand, and formic acid as the capping molecule;
(b) Zr⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(c) Zr⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(d) Hf⁴⁺ as the metal ion, benzene-1,3,5-tribenzoate as the organic ligand, and formic acid as the capping molecule;
(e) Hf⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(f) Hf⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(g) Zr⁺ as the metal ion and benzene-1,3,5-tribenzoate as the organic ligand;
(h) Zr⁴⁺ as the metal ion and 5′-(4-carboxyphenyl)-2′-methyl[1,1′:3′,1″-terphenyl]-4,4″-dicarboxylicacid as the organic ligand;
(i) Zr⁴⁺ as the metal ion and 2′-amino-5′-(4-carboxyphenyl)-[1,1′:3′,1″-terphenyl]-4,4″-dicarboxylic acid as the organic ligand;
(j) Hf⁴⁺ as the metal ion and benzene-1,3,5-tribenzoate as the organic ligand;
(k) Hf⁴⁺ as the metal ion and 5′-(4-carboxyphenyl)-2′-methyl[1,1′:3′,1″-terphenyl]-4,4″-dicarboxylicacid as the organic ligand; or
(l) Hf⁴⁺ as the metal ion and 2′-amino-5′-(4-carboxyphenyl)-[1,1′:3′,1″-terphenyl]-4,4″-dicarboxylic acid as the organic ligand.

18. (canceled)

19. (canceled)

20. The composition according to claim 11, wherein the composition is in the form of a gel or a film.

21. (canceled)

22. A method of making a gel from a suspension as described in claim 9, wherein the method comprises the steps of:
(a) providing the suspension as described in claim 9; and
(b) removing the water and/or THF to evaporate to form a gel.

23. (canceled)

24. (canceled)

25. (canceled)

26. A method of making a sol claim 10, wherein the method comprises the steps of:
(a) providing a gel as described in claim 20; and
(b) adding a solvent to the gel to form the sol,
wherein the sol comprises the solvent and MOF nanosheets having an area of greater than or equal to 10.000 μm².

27. (canceled)

28. The sol according to claim 10, wherein the MOF in the MOF nanosheets is selected from one or more of NUS-8 and/or an analogue thereof, where each MOF is formed from:
(a) a metal ion and an organic ligand: or
(b) a metal ion and an organic ligand and a capping molecule.

29. The composition according to claim 11, wherein the MOF in the MOF nanosheets is selected from one or more of NUS-8 and/or an analogue thereof, where each MOF is formed from:
(a) a metal ion and an organic ligand: or
(b) a metal ion and an organic ligand and a capping molecule.

30. The sol according to claim 10, wherein the MOF is NUS-8 or an analogue thereof having:
(a) Zr⁺ as the metal ion, benzene-1,3,5-tribenzoate as the organic ligand, and formic acid as the capping molecule;
(b) Zr⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(c) Zr⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(d) Hf⁴⁺ as the metal ion, benzene-1,3,5-tribenzoate as the organic ligand, and formic acid as the capping molecule;
(e) Hf⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(f) Hf⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(g) Zr⁴⁺ as the metal ion and benzene-1,3,5-tribenzoate as the organic ligand;
(h) Zr⁴⁺ as the metal ion and 5′-(4-carboxyphenyl)-2′-methyl[1,1′:3′,1″-terphenyl]-4,4″-dicarboxylicacid as the organic ligand;
(i) Zr⁴⁺ as the metal ion and 2′-amino-5′-(4-carboxyphenyl)-[1,1′:3′,1″-terphenyl]-4,4″-dicarboxylic acid as the organic ligand;
(j) Hf⁴⁺ as the metal ion and benzene-1,3,5-tribenzoate as the organic ligand;
(k) Hf⁴⁺ as the metal ion and 5′-(4-carboxyphenyl)-2′-methyl[1,1′:3′,1″-terphenyl]-4,4″-dicarboxylicacid as the organic ligand; or
(1) Hf⁴⁺ as the metal ion and 2′-amino-5′-(4-carboxyphenyl)-[1,1′:3′,1″-terphenyl]-4,4″-dicarboxylic acid as the organic ligand.

31. The composition according to claim 11, wherein the MOF is NUS-8 or an analogue thereof having:
(a) Zr⁺ as the metal ion, benzene-1,3,5-tribenzoate as the organic ligand, and formic acid as the capping molecule;
(b) Zr⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(c) Zr⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(d) Hf⁴⁺ as the metal ion, benzene-1,3,5-tribenzoate as the organic ligand, and formic acid as the capping molecule;
(e) Hf⁴⁺ as the metal ion, 5′-(4-carboxyphenyl)-2′-methyl[1,1′:3′,1″-terphenyl]-4,4″-dicarboxylicacid as the organic ligand, and formic acid as the capping molecule;
(f) Hf⁴⁺ as the metal ion, 2′-amino-5′-(4-carboxyphenyl)-[1,1′:3′,1″-terphenyl]-4,4″-dicarboxylic acid as the organic ligand, and formic acid as the capping molecule;
(g) Zr⁴⁺ as the metal ion and benzene-1,3,5-tribenzoate as the organic ligand;
(h) Zr⁴⁺ as the metal ion and 5′-(4-carboxyphenyl)-2′-methyl[1,1′:3′,1″-terphenyl]-4,4″-dicarboxylicacid as the organic ligand;
(i) Zr⁴⁺ as the metal ion and 2′-amino-5′-(4-carboxyphenyl)-[1,1′:3′,1″-terphenyl]-4,4″-dicarboxylic acid as the organic ligand;
(j) Hf⁴⁺ as the metal ion and benzene-1,3,5-tribenzoate as the organic ligand;
(k) Hf⁴⁺ as the metal ion and 5′-(4-carboxyphenyl)-2′-methyl[1,1′:3′,1″-terphenyl]-4,4″-dicarboxylicacid as the organic ligand; or
(l) Hf⁴⁺ as the metal ion and 2′-amino-5′-(4-carboxyphenyl)-[1,1′:3′,1″-terphenyl]-4,4″-dicarboxylic acid as the organic ligand.
